SEQUENCE LISTING (48 sequences)

SEQ ID NO: 1 - DNA, 96 bp, Arabidopsis thaliana
  CDS (1)..(96)
atgagtaaacggaatccgaagattctgaagatttttctgtatatgttacttctcaactctctctttctcatcatctacttcgtttttcactcatcg

SEQ ID NO: 2 - protein, 32 aa, Arabidopsis thaliana
MSKRNPKILKIFLYMLLLNSLFLIIYFVFHSS

SEQ ID NO: 3 - DNA, 186 bp, Arabidopsis thaliana
  CDS (1)..(186)
atgggtgttttctcgaatcttcgaggacccagagccggagctacccacgatgaatttccggcgaccaatggctctccttcgtcttcttcttctccatcttcatcaatcaagcgaaaattatcgaatttgttaccactctgcgttgctctggtagttatcgctgagatcgggtttctgggtcggctc

SEQ ID NO: 4 - protein, 62 aa, Arabidopsis thaliana
MGVFSNLRGPRAGATHDEFPATNGSPSSSSSPSSSIKRKLSNLLPLCVALVVIAEIGFLGRL

SEQ ID NO: 5 - DNA, 1527 bp, homo sapiens
  CDS (1)..(1527)
caaaatgggaaagaaaaactgattgcttatcaacgagaattccttgctttgaaagaacgtcttcgaatagctgaacacagaatctcacagcgctcttctgaattaaatacgattgtgcaacagttcaagcgtgtaggagcagaaacaaatggaagtaaggatgcgttgaataagttttcagataataccctaaagctgttaaaggagttaacaagcaaaaaatctcttcaagtgccaagtatttattatcatttgcctcatttattgaaaaatgaaggaagtcttcaacctgctgtacagattggcaacggaagaacaggagtttcaatagtcatgggcattcccacagtgaagagagaagttaaatcttacctcatagaaactcttcattcccttattgataacctgtatcctgaagagaagttggactgtgttatagtagtcttcataggagagacagatattgattatgtacatggtgttgtagccaacctggagaaagaattttctaaagaaatcagttctggcttggtggaagtcatatcaccccctgaaagctattatcctgacttgacaaacctaaaggagacatttggagactccaaagaaagagtaagatggagaacaaagcaaaacctagattactgttttctaatgatgtatgctcaagaaaagggcatatattacattcagcttgaagatgatattattgtcaaacaaaattattttaataccataaaaaattttgcacttcaactttcttctgaggaatggatgattctagagttttcccagctgggcttcattggtaaaatgtttcaagcgccggatcttactctgattgtagaattcatattcatgttttacaaggagaaacccattgattggctcctggaccatattctctgggtgaaagtctgcaaccctgaaaaagatgcaaaacattgtgatagacagaaagcaaatctgcgaattcgcttcagaccttcccttttccaacatgttggtctgcactcatcactatcaggaaaaatccaaaaactcacggataaagattatatgaaaccattacttcttaaaatccatgtaaacccacctgcggaggtatctacttccttgaaggtctaccaagggcatacgctggagaaaacttacatgggagaggatttcttctgggctatcacaccgatagctggagactacatcttgtttaaatttgataaaccagtcaatgtagaaagttatttgttccatagcggcaaccaagaacatcctggagatattctgctaaacacaactgtggaagttttgccttttaagagtgaaggtttggaaataagcaaagaaaccaaagacaaacgattagaagatggctatttcagaataggaaaatttgagaatggtgttgcagaaggaatggtggatccaagtctcaatcccatttcagcctttcgactttcagttattcagaattctgctgtttgggccattcttaatgagattcatattaaaaaagccaccaactga

SEQ ID NO: 6 - protein, 508 aa, homo sapiens
QNGKEKLIAYQREFLALKERLRIAEHRISQRSSELNTIVQQFKRVGAETNGSKDALNKFSDNTLKLLKELTSKKSLQVPSIYYHLPHLLKNEGSLQPAVQIGNGRTGVSIVMGIPTVKREVKSYLIETLHSLIDNLYPEEKLDCVIVVFIGETDIDYVHGVVANLEKEFSKEISSGLVEVISPPESYYPDLTNLKETFGDSKERVRWRTKQNLDYCFLMMYAQEKGIYYIQLEDDIIVKQNYFNTIKNFALQLSSEEWMILEFSQLGFIGKMFQAPDLTLIVEFIFMFYKEKPIDWLLDHILWVKVCNPEKDAKHCDRQKANLRIRFRPSLFQHVGLHSSLSGKIQKLTDKDYMKPLLLKIHVNPPAEVSTSLKVYQGHTLEKTYMGEDFFWAITPIAGDYILFKFDKPVNVESYLFHSGNQEHPGDILLNTTVEVLPFKSEGLEISKETKDKRLEDGYFRIGKFENGVAEGMVDPSLNPISAFRLSVIQNSAVWAILNEIHIKKATN

SEQ ID NO: 7 - DNA, 1548 bp, homo sapiens
  CDS (1)..(1548)
gacgttgtggacgtttaccagcgggagttcctggcgctgcgcgatcggttgcacgcagctgagcaggagagcctcaagcgctccaaggagctcaacctggtgctggacgagatcaagagggccgtgtcagaaaggcaggcgctgcgagacggagacggcaatcgcacctggggccgcctaacagaggacccccgattgaagccgtggaacggctcacaccggcacgtgctgcacctgcccaccgtcttccatcacctgccacacctgctggccaaggagagcagtctgcagcccgcggtgcgcgtgggccagggccgcaccggagtgtcggtggtgatgggcatcccgagcgtgcggcgcgaggtgcactcgtacctgactgacactctgcactcgctcatctccgagctgagcccgcaggagaaggaggactcggtcatcgtggtgctgatcgccgagactgactcacagtacacttcggcagtgacagagaacatcaaggccttgttccccacggagatccattctgggctcctggaggtcatctcaccctccccccacttctaccctgacttctcccgcctccgagagtcctttggggaccccaaggagagagtcaggtggaggaccaaacagaacctcgattactgcttcctcatgatgtacgcgcagtccaaaggcatctactacgtgcagctggaggatgacatcgtggccaagcccaactacctgagcaccatgaagaactttgcactgcagcagccttcagaggactggatgatcctggagttctcccagctgggcttcattggtaagatgttcaagtcgctggacctgagcctgattgtagagttcattctcatgttctaccgggacaagcccatcgactggctcctggaccatattctgtgggtgaaagtctgcaaccccgagaaggatgcgaagcactgtgaccggcagaaagccaacctgcggatccgcttcaaaccgtccctcttccagcacgtgggcactcactcctcgctggctggcaagatccagaaactgaaggacaaagactttggaaagcaggcgctgcggaaggagcatgtgaacccgccagcagaggtgagcacgagcctgaagacataccagcacttcaccctggagaaagcctacctgcgcgaggacttcttctgggccttcacccctgccgcgggggacttcatccgcttccgcttcttccaacctctaagactggagcggttcttcttccgcagtgggaacatcgagcacccggaggacaagctcttcaacacgtctgtggaggtgctgcccttcgacaaccctcagtcagacaaggaggccctgcaggagggccgcaccgccaccctccggtaccctcggagccccgacggctacctccagatcggctccttctacaagggagtggcagagggagaggtggacccagccttcggccctctggaagcactgcgcctctcgatccagacggactcccctgtgtgggtgattctgagcgagatcttcctgaaaaaggccgactaa

SEQ ID NO: 8 - protein, 515 aa, homo sapiens
DVVDVYQREFLALRDRLHAAEQESLKRSKELNLVLDEIKRAVSERQALRDGDGNRTWGRLTEDPRLKPWNGSHRHVLHLPTVFHHLPHLLAKESSLQPAVRVGQGRTGVSVVMGIPSVRREVHSYLTDTLHSLISELSPQEKEDSVIVVLIAETDSQYTSAVTENIKALFPTEIHSGLLEVISPSPHFYPDFSRLRESFGDPKERVRWRTKQNLDYCFLMMYAQSKGIYYVQLEDDIVAKPNYLSTMKNFALQQPSEDWMILEFSQLGFIGKMFKSLDLSLIVEFILMFYRDKPIDWLLDHILWVKVCNPEKDAKHCDRQKANLRIRFKPSLFQHVGTHSSLAGKIQKLKDKDFGKQALRKEHVNPPAEVSTSLKTYQHFTLEKAYLREDFFWAFTPAAGDFIRFRFFQPLRLERFFFRSGNIEHPEDKLFNTSVEVLPFDNPQSDKEALQEGRTATLRYPRSPDGYLQIGSFYKGVAEGEVDPAFGPLEALRLSIQTDSPVWVILSEIFLKKAD

SEQ ID NO: 9 - DNA, 2109 bp, Artificial
  catalytic domain of Homo sapiens GnT-Va sequence which is codon optimized for Nicotiana benthamiana
cctgagtcatcttctatgctcagagagcagatccttgatctctctaagagatatatcaaggctctcgctgaagagaacagaaacgtggtggatggaccttatgctggtgttatgactgcttacgatctcaagaaaactcttgctgtgctcctcgataacatccttcagagaatcggaaagctcgagtctaaagtggataacctcgtggttaacggaactggaactaactctacaaactctactactgctgtgccttctcttgttgctctcgagaagatcaacgtggctgatatcatcaatggtgctcaagagaagtgtgttctccctcctatggatggatatcctcactgtgagggaaagatcaagtggatgaaggatatgtggagatctgatccttgctacgctgattacggtgttgatggatctacttgctctttcttcatctacctctcagaggttgaaaactggtgtcctcatcttccttggagagctaagaacccttacgaagaggctgatcacaattctctcgctgagatcagaacagatttcaacatcctctactctatgatgaagaagcacgaagagttcagatggatgagactcagaatcagaagaatggctgacgcttggatacaagcaatcaagtcacttgctgagaagcagaacctcgagaagagaaagagaaagaaggttctcgttcatcttggactcctcacaaaagagtctggattcaagatcgctgagactgctttttctggtggacctcttggagaacttgttcagtggagtgatctcatcacttcactctacctcctcggacacgatattagaatctctgcttctctcgcagagcttaaagaaatcatgaagaaagtggtgggtaacagatctggatgtcctactgtgggagatagaatcgtggagcttatctacatcgatatcgtgggtcttgctcagttcaaaaagactctcggaccttcttgggttcactaccagtgcatgcttagagtgctcgattctttcggaactgagcctgagtttaaccacgctaactacgctcaatctaagggacataagacaccttggggaaagtggaaccttaaccctcagcagttctacactatgttccctcacactcctgataactcattcctcggattcgttgttgagcagcacctcaactcttcagatatccaccacatcaacgagatcaagagacagaaccagtctcttgtgtacggaaaagtggattctttctggaagaacaagaagatctacctcgatatcatccacacttacatggaagttcacgctactgtgtacggatcttctactaagaacatcccttcttacgtgaagaaccacggaatcctctcaggaagagatcttcagttcctcctcagagagactaagctcttcgttggtcttggatttccttatgagggacctgctcctcttgaagctatcgctaacggatgtgctttcctcaaccctaagttcaaccctcctaagtcatctaagaacactgatttcttcatcggaaagcctactctcagagagcttacttctcagcatccttacgctgaggttttcatcggtagacctcatgtttggactgtggatctcaacaaccaggaagaggttgaggacgctgttaaggctatcctcaaccagaaaatcgaaccttacatgccttacgagttcacttgtgagggaatgctccagaggatcaacgctttcatcgagaagcaggatttctgtcatggacaggttatgtggcctcctctttctgctcttcaggttaagcttgctgaacctggacaatcttgcaagcaagtgtgccaagagtctcagcttatctgcgagccttctttcttccagcatctcaacaaggataaggatatgctcaagtacaaggtgacatgccagtcatctgagcttgctaaggatatcctcgtgccttctttcgatcctaagaacaagcactgtgtgttccagggtgatcttcttcttttctcttgcgctggtgctcatcctagacatcagagagtttgcccttgcagagatttcatcaagggacaagtggctctctgtaaggattgcctctga

SEQ ID NO: 10 - protein, 702 aa, Artificial
  Synthetic Construct
PESSSMLREQILDLSKRYIKALAEENRNVVDGPYAGVMTAYDLKKTLAVLLDNILQRIGKLESKVDNLVVNGTGTNSTNSTTAVPSLVALEKINVADIINGAQEKCVLPPMDGYPHCEGKIKWMKDMWRSDPCYADYGVDGSTCSFFIYLSEVENWCPHLPWRAKNPYEEADHNSLAEIRTDFNILYSMMKKHEEFRWMRLRIRRMADAWIQAIKSLAEKQNLEKRKRKKVLVHLGLLTKESGFKIAETAFSGGPLGELVQWSDLITSLYLLGHDIRISASLAELKEIMKKVVGNRSGCPTVGDRIVELIYIDIVGLAQFKKTLGPSWVHYQCMLRVLDSFGTEPEFNHANYAQSKGHKTPWGKWNLNPQQFYTMFPHTPDNSFLGFVVEQHLNSSDIHHINEIKRQNQSLVYGKVDSFWKNKKIYLDIIHTYMEVHATVYGSSTKNIPSYVKNHGILSGRDLQFLLRETKLFVGLGFPYEGPAPLEAIANGCAFLNPKFNPPKSSKNTDFFIGKPTLRELTSQHPYAEVFIGRPHVWTVDLNNQEEVEDAVKAILNQKIEPYMPYEFTCEGMLQRINAFIEKQDFCHGQVMWPPLSALQVKLAEPGQSCKQVCQESQLICEPSFFQHLNKDKDMLKYKVTCQSSELAKDILVPSFDPKNKHCVFQGDLLLFSCAGAHPRHQRVCPCRDFIKGQVALCKDCL

SEQ ID NO: 11 - DNA, 1623 bp, Artificial
  nucleotide sequence of hybrid GnT-IVa (xylosyltransferase localization signal derived from A. thaliana fused to catalytic domain of GnT-IVa derived from Homo sapiens), codon optimized for Nicotiana benthamiana
atgtccaaacggaacccgaaaattctcaagattttcctttacatgctgcttctaaactccttgttcctcattatatacttcgtcttccattctagccaaaatggaaaggaaaagctgattgcataccaaagagagtttcttgcactaaaggaaaggcttagaattgctgaacacagaatttctcagagatcctctgaactgaatacgattgttcagcaatttaaacgcgtaggtgctgaaactaatggttctaaggatgctctaaacaagtttagcgacaataccttgaagttgctcaaggaattgacctctaaaaagtcacttcaagtccctagtatctactatcatcttccacacttgcttaagaatgaaggatcacttcaaccagcagtacaaattggtaatggtagaacaggagtttctatagtcatgggtattcctactgttaagagggaagttaagagttacctcattgagacattgcactcacttatcgacaatctttatcctgaagagaaacttgattgcgtaatcgttgttttcattggagagactgacatagattatgttcatggtgtcgttgcaaatctcgaaaaggaatttagtaaggagatctcctctggtcttgttgaagttatatctccacctgagtcttattatccggatcttaccaacctaaaggaaacttttggtgactcaaaggaaagagttagatggagaacaaagcagaacctcgattattgtttcctgatgatgtatgctcaggaaaagggaatctactacattcaacttgaggacgatataatcgtcaagcagaactactttaacacgatcaagaactttgccttgcaacttagttctgaagaatggatgatccttgagtttagtcaacttggattcattggcaagatgtttcaagcacctgatctaactctcatagtggagtttatcttcatgttctacaaggagaaacccattgattggctacttgatcacatactttgggttaaggtttgtaacccagaaaaggatgcaaagcattgtgatagacaaaaggctaatcttcgaatccgttttagaccatcattgttccaacatgttggattgcattcatctttgtctgggaagattcaaaaacttaccgataaggactatatgaaaccactgcttctgaagattcatgtaaatcctccagccgaagtttctacaagtcttaaggtatatcagggtcatacactcgaaaagacttatatgggagaggattttttctgggctattactcctattgctggagattatatcctgttcaagtttgacaagcctgtgaatgttgagagttacctttttcactctggtaatcaagaacatccaggtgatatactgcttaacacaacggttgaagttcttccttttaagtcagaaggcttggaaatttcaaaggagactaaggacaagagacttgaagatggttatttccgaataggcaagtttgaaaatggtgttgctgagggtatggttgatccttctcttaatcccatatcagcttttcgtctcagtgttatccaaaattctgcagtttgggctattctgaatgagattcacatcaagaaggctacaaattga

SEQ ID NO: 12 - protein, 540 aa, Artificial
  Synthetic Construct
MSKRNPKILKIFLYMLLLNSLFLIIYFVFHSSQNGKEKLIAYQREFLALKERLRIAEHRISQRSSELNTIVQQFKRVGAETNGSKDALNKFSDNTLKLLKELTSKKSLQVPSIYYHLPHLLKNEGSLQPAVQIGNGRTGVSIVMGIPTVKREVKSYLIETLHSLIDNLYPEEKLDCVIVVFIGETDIDYVHGVVANLEKEFSKEISSGLVEVISPPESYYPDLTNLKETFGDSKERVRWRTKQNLDYCFLMMYAQEKGIYYIQLEDDIIVKQNYFNTIKNFALQLSSEEWMILEFSQLGFIGKMFQAPDLTLIVEFIFMFYKEKPIDWLLDHILWVKVCNPEKDAKHCDRQKANLRIRFRPSLFQHVGLHSSLSGKIQKLTDKDYMKPLLLKIHVNPPAEVSTSLKVYQGHTLEKTYMGEDFFWAITPIAGDYILFKFDKPVNVESYLFHSGNQEHPGDILLNTTVEVLPFKSEGLEISKETKDKRLEDGYFRIGKFENGVAEGMVDPSLNPISAFRLSVIQNSAVWAILNEIHIKKATN

SEQ ID NO: 13 - DNA, 1713 bp, Artificial
  nucleotide sequence of hybrid GnT-IVa (fucosyltransferase B localization signal derived from A. thaliana fused to catalytic domain of GnT-IVa derived from Homo sapiens), codon optimized for Nicotiana benthamiana
atgggagtcttttcaaacttaagaggtccaagagctggtgcaacacatgatgaatttcctgctactaatggttctccttctagttcttcatctcccagttcttctattaagcgtaagctctctaatcttcttccgctttgtgttgctcttgttgtgattgctgaaattggctttctaggacgacttcaaaatggaaaggaaaagctgattgcataccaaagagagtttcttgcactaaaggaaaggcttagaattgctgaacacagaatttctcagagatcctctgaactgaatacgattgttcagcaatttaaacgcgtaggtgctgaaactaatggttctaaggatgctctaaacaagtttagcgacaataccttgaagttgctcaaggaattgacctctaaaaagtcacttcaagtccctagtatctactatcatcttccacacttgcttaagaatgaaggatcacttcaaccagcagtacaaattggtaatggtagaacaggagtttctatagtcatgggtattcctactgttaagagggaagttaagagttacctcattgagacattgcactcacttatcgacaatctttatcctgaagagaaacttgattgcgtaatcgttgttttcattggagagactgacatagattatgttcatggtgtcgttgcaaatctcgaaaaggaatttagtaaggagatctcctctggtcttgttgaagttatatctccacctgagtcttattatccggatcttaccaacctaaaggaaacttttggtgactcaaaggaaagagttagatggagaacaaagcagaacctcgattattgtttcctgatgatgtatgctcaggaaaagggaatctactacattcaacttgaggacgatataatcgtcaagcagaactactttaacacgatcaagaactttgccttgcaacttagttctgaagaatggatgatccttgagtttagtcaacttggattcattggcaagatgtttcaagcacctgatctaactctcatagtggagtttatcttcatgttctacaaggagaaacccattgattggctacttgatcacatactttgggttaaggtttgtaacccagaaaaggatgcaaagcattgtgatagacaaaaggctaatcttcgaatccgttttagaccatcattgttccaacatgttggattgcattcatctttgtctgggaagattcaaaaacttaccgataaggactatatgaaaccactgcttctgaagattcatgtaaatcctccagccgaagtttctacaagtcttaaggtatatcagggtcatacactcgaaaagacttatatgggagaggattttttctgggctattactcctattgctggagattatatcctgttcaagtttgacaagcctgtgaatgttgagagttacctttttcactctggtaatcaagaacatccaggtgatatactgcttaacacaacggttgaagttcttccttttaagtcagaaggcttggaaatttcaaaggagactaaggacaagagacttgaagatggttatttccgaataggcaagtttgaaaatggtgttgctgagggtatggttgatccttctcttaatcccatatcagcttttcgtctcagtgttatccaaaattctgcagtttgggctattctgaatgagattcacatcaagaaggctacaaattga

SEQ ID NO: 14 - protein, 570 aa, Artificial
  Synthetic Construct
MGVFSNLRGPRAGATHDEFPATNGSPSSSSSPSSSIKRKLSNLLPLCVALVVIAEIGFLGRLQNGKEKLIAYQREFLALKERLRIAEHRISQRSSELNTIVQQFKRVGAETNGSKDALNKFSDNTLKLLKELTSKKSLQVPSIYYHLPHLLKNEGSLQPAVQIGNGRTGVSIVMGIPTVKREVKSYLIETLHSLIDNLYPEEKLDCVIVVFIGETDIDYVHGVVANLEKEFSKEISSGLVEVISPPESYYPDLTNLKETFGDSKERVRWRTKQNLDYCFLMMYAQEKGIYYIQLEDDIIVKQNYFNTIKNFALQLSSEEWMILEFSQLGFIGKMFQAPDLTLIVEFIFMFYKEKPIDWLLDHILWVKVCNPEKDAKHCDRQKANLRIRFRPSLFQHVGLHSSLSGKIQKLTDKDYMKPLLLKIHVNPPAEVSTSLKVYQGHTLEKTYMGEDFFWAITPIAGDYILFKFDKPVNVESYLFHSGNQEHPGDILLNTTVEVLPFKSEGLEISKETKDKRLEDGYFRIGKFENGVAEGMVDPSLNPISAFRLSVIQNSAVWAILNEIHIKKATN

SEQ ID NO: 15 - DNA, 1644 bp, Artificial
  nucleotide sequence of hybrid GnT-IVb (xylosyltransferase localization signal derived from A. thaliana fused to catalytic domain of GnT-IVb derived from Homo sapiens), codon optimized for Nicotiana benthamiana
atgagtaagcgaaatcccaagatcctgaagattttcttgtacatgctcctgttgaactctcttttcctcatcatatacttcgtatttcactcatctgacgttgtagatgtataccaacgcgaatttcttgctcttagagatagacttcatgctgcagaacaagaatccctaaagcgctccaaagaacttaatcttgtcctagatgagattaagcgtgctgtgtctgaaagacaagctcttagagacggagatggtaatagaacatggggaagacttacagaagatcctaggcttaagccatggaatggatctcatagacatgtactccacttaccaacagttttccatcatcttcctcacctacttgcaaaagaatcttcattgcaacctgctgttagagttggtcaaggtagaactggagtttcagtagttatgggtattccaagcgttagaagagaagttcactcttatctcactgataccctccattctttgatttctgagttgtcaccacaagagaaggaagattctgtaattgttgtccttatcgctgagactgattctcaatatacttctgccgttactgagaacatcaaagcacttttcccaactgaaatacactctggtttactggaggtaatatcaccttctcctcatttttacccagactttagtaggttgcgagaatcttttggagatcctaaagaaagagtcagatggaggactaagcaaaatcttgactactgctttcttatgatgtatgcccaatccaagggaatctattatgttcagcttgaggatgacattgttgctaaacccaactacctgagtaccatgaagaattttgccttacagcaaccaagcgaagattggatgatactggaattttcccagcttggtttcattggcaagatgttcaagagccttgatcttagtctgattgtcgagttcatccttatgttctaccgagataagccaatagattggttgcttgatcacattctctgggttaaggtttgtaatccggaaaaggatgctaagcattgtgatagacagaaggctaacttgcgtattagattcaaaccgtcactctttcaacatgttggaactcatagttctctcgctggtaagatccaaaagctgaaggataaggattttgggaaacaagctctgagaaaggaacatgttaatcctccagcagaagtttctacttcactaaagacgtaccagcactttacacttgagaaggcttatcttagggaggactttttttgggcatttacacctgcagctggtgatttcattagattccggtttttccaaccactaagacttgaacgctttttctttcgttctggcaatatagagcatcctgaagacaagttgtttaacacatccgttgaagtgttgccttttgataaccctcaatctgacaaagaagcacttcaagaaggtagaacagcaacacttaggtatcctagatcacccgacggttatcttcaaatcggatcattttataagggagttgcagaaggtgaagttgatccagcttttggtcctttagaagctctaaggctttctattcagactgattctcctgtctgggttattcttagcgaaattttcctgaagaaggccgactga

SEQ ID NO: 16 - protein, 547 aa, Artificial
  Synthetic Construct
MSKRNPKILKIFLYMLLLNSLFLIIYFVFHSSDVVDVYQREFLALRDRLHAAEQESLKRSKELNLVLDEIKRAVSERQALRDGDGNRTWGRLTEDPRLKPWNGSHRHVLHLPTVFHHLPHLLAKESSLQPAVRVGQGRTGVSVVMGIPSVRREVHSYLTDTLHSLISELSPQEKEDSVIVVLIAETDSQYTSAVTENIKALFPTEIHSGLLEVISPSPHFYPDFSRLRESFGDPKERVRWRTKQNLDYCFLMMYAQSKGIYYVQLEDDIVAKPNYLSTMKNFALQQPSEDWMILEFSQLGFIGKMFKSLDLSLIVEFILMFYRDKPIDWLLDHILWVKVCNPEKDAKHCDRQKANLRIRFKPSLFQHVGTHSSLAGKIQKLKDKDFGKQALRKEHVNPPAEVSTSLKTYQHFTLEKAYLREDFFWAFTPAAGDFIRFRFFQPLRLERFFFRSGNIEHPEDKLFNTSVEVLPFDNPQSDKEALQEGRTATLRYPRSPDGYLQIGSFYKGVAEGEVDPAFGPLEALRLSIQTDSPVWVILSEIFLKKAD

SEQ ID NO: 17 - DNA, 1713 bp, Artificial; identical to SEQ ID NO: 13
  nucleotide sequence of hybrid GnT-IVb (fucosyltransferase B localization signal derived from A. thaliana fused to catalytic domain of GnT-IVb derived from Homo sapiens), codon optimized for Nicotiana benthamiana

SEQ ID NO: 18 - protein, 570 aa, Artificial; identical to SEQ ID NO: 14
  Synthetic Construct

SEQ ID NO: 19 - DNA, 2205 bp, Artificial
  nucleotide sequence of hybrid GnT-Va (xylosyltransferase localization signal derived from A. thaliana fused to catalytic domain of GnT-Va derived from Homo sapiens), codon optimized for Nicotiana benthamiana
atgtccaagagaaaccccaagatactgaagatcttcctatatatgctcctgcttaatagcctgtttctgatcatttatttcgtcttccactctagtccagaatcatcttcaatgcttagagagcaaatcctcgatttgtccaagagatacattaaggcacttgctgaagagaacagaaatgttgttgatggtccttatgcaggagttatgactgcctatgatcttaagaagacattagccgttctgcttgacaacattctacagagaataggcaagttggagagtaaggttgataatcttgtggtcaatggaacgggaacaaattcaaccaatagcactacagctgttccttctttagttgctcttgagaagatcaatgttgccgacataatcaatggtgcacaagaaaagtgtgtacttcctcctatggatggttaccctcattgtgaaggaaagatcaagtggatgaaagatatgtggagatctgatccttgttatgctgattatggtgtggatggaagtacctgctcatttttcatctatctgtctgaagttgagaattggtgtcctcatcttccttggagagctaagaatccatacgaagaggctgatcataattcattggctgaaatcaggacggacttcaatatcctatactctatgatgaagaagcacgaagagttcagatggatgagacttagaattagaagaatggcagacgcgtggatacaagctattaagagtcttgctgagaagcaaaacctagaaaagaggaagcgaaagaaggttcttgtacatcttggtttgcttaccaaggagtctggttttaaaattgcagaaactgccttttctggaggtccacttggtgaattagtacagtggtcagacttgataacatctctctacttgcttggccatgacattagaatttctgctagtcttgctgagttgaaggaaatcatgaagaaggttgttggcaatagatctggatgtcctactgttggtgatcgcattgttgaactgatttatattgatatagtcggacttgcccagtttaagaaaactttaggtcctagttgggtacattaccaatgtatgctgagagtactcgattcttttggtactgaacctgaatttaaccacgctaactatgctcaaagtaaaggtcataagacaccatggggaaagtggaatcttaacccccaacaattctacacaatgtttcctcacactccggataatagtttccttggctttgttgttgaacagcaccttaattcctctgacatacaccacattaatgaaattaagaggcagaaccaaagcctcgtttatggaaaagttgactccttttggaagaacaagaagatctatctggatataattcatacgtacatggaagttcatgctactgtttacgggtcatctaccaagaatattccctcctatgtcaagaatcatggtatactttctggaagagacttgcagttcttgcttagagaaaccaaactttttgtcggtcttggttttccttatgaaggtcctgcaccacttgaagctattgcaaatggatgtgcttttctcaacccaaagttcaatcctccaaagtcaagcaagaacaccgatttctttattggcaagcctacactaagagaacttacttcacaacacccttatgccgaagtttttattggaagaccacatgtttggacagtcgatttgaacaatcaagaggaagttgaggatgcagttaaggctatccttaaccagaagattgaaccatacatgccctacgaatttacatgtgaaggtatgttgcagcggattaacgcattcattgaaaagcaggacttttgtcatggtcaagttatgtggccacctctttcagctcttcaagtaaagttagcagaacctggtcaatcttgtaagcaagtttgtcaagagtcacagcttatttgcgaaccttcttttttccagcacctcaataaggataaggatatgctcaagtacaaggttacttgccaatcttctgaacttgcaaaggatatactcgttccatcttttgacccaaagaataagcattgcgtcttccaaggtgatcttctattgtttagttgtgctggagcacatcctagacatcaaagagtatgtccatgcagagatttcataaagggtcaagtagctctatgcaaggattgcctttga

SEQ ID NO: 20 - protein, 734 aa, Artificial
  Synthetic Construct
MSKRNPKILKIFLYMLLLNSLFLIIYFVFHSSPESSSMLREQILDLSKRYIKALAEENRNVVDGPYAGVMTAYDLKKTLAVLLDNILQRIGKLESKVDNLVVNGTGTNSTNSTTAVPSLVALEKINVADIINGAQEKCVLPPMDGYPHCEGKIKWMKDMWRSDPCYADYGVDGSTCSFFIYLSEVENWCPHLPWRAKNPYEEADHNSLAEIRTDFNILYSMMKKHEEFRWMRLRIRRMADAWIQAIKSLAEKQNLEKRKRKKVLVHLGLLTKESGFKIAETAFSGGPLGELVQWSDLITSLYLLGHDIRISASLAELKEIMKKVVGNRSGCPTVGDRIVELIYIDIVGLAQFKKTLGPSWVHYQCMLRVLDSFGTEPEFNHANYAQSKGHKTPWGKWNLNPQQFYTMFPHTPDNSFLGFVVEQHLNSSDIHHINEIKRQNQSLVYGKVDSFWKNKKIYLDIIHTYMEVHATVYGSSTKNIPSYVKNHGILSGRDLQFLLRETKLFVGLGFPYEGPAPLEAIANGCAFLNPKFNPPKSSKNTDFFIGKPTLRELTSQHPYAEVFIGRPHVWTVDLNNQEEVEDAVKAILNQKIEPYMPYEFTCEGMLQRINAFIEKQDFCHGQVMWPPLSALQVKLAEPGQSCKQVCQESQLICEPSFFQHLNKDKDMLKYKVTCQSSELAKDILVPSFDPKNKHCVFQGDLLLFSCAGAHPRHQRVCPCRDFIKGQVALCKDCL

SEQ ID NO: 21 - DNA, 2295 bp, Artificial
  nucleotide sequence of hybrid GnT-Va (fucosyltransferase B localization signal derived from A. thaliana fused to catalytic domain of GnT-Va derived from Homo sapiens), codon optimized for Nicotiana benthamiana
atgggagttttcagcaatctcagaggtccaagagctggtgctactcatgatgaatttcctgctacaaatggttctccttcttcttcttcttctccttcttcttctattaagaggaaactttctaatcttcttcctctttgtgttgctcttgttgttattgctgaaatcggttttcttggtagacttccagaatcatcttcaatgcttagagagcaaatcctcgatttgtccaagagatacattaaggcacttgctgaagagaacagaaatgttgttgatggtccttatgcaggagttatgactgcctatgatcttaagaagacattagccgttctgcttgacaacattctacagagaataggcaagttggagagtaaggttgataatcttgtggtcaatggaacgggaacaaattcaaccaatagcactacagctgttccttctttagttgctcttgagaagatcaatgttgccgacataatcaatggtgcacaagaaaagtgtgtacttcctcctatggatggttaccctcattgtgaaggaaagatcaagtggatgaaagatatgtggagatctgatccttgttatgctgattatggtgtggatggaagtacctgctcatttttcatctatctgtctgaagttgagaattggtgtcctcatcttccttggagagctaagaatccatacgaagaggctgatcataattcattggctgaaatcaggacggacttcaatatcctatactctatgatgaagaagcacgaagagttcagatggatgagacttagaattagaagaatggcagacgcgtggatacaagctattaagagtcttgctgagaagcaaaacctagaaaagaggaagcgaaagaaggttcttgtacatcttggtttgcttaccaaggagtctggttttaaaattgcagaaactgccttttctggaggtccacttggtgaattagtacagtggtcagacttgataacatctctctacttgcttggccatgacattagaatttctgctagtcttgctgagttgaaggaaatcatgaagaaggttgttggcaatagatctggatgtcctactgttggtgatcgcattgttgaactgatttatattgatatagtcggacttgcccagtttaagaaaactttaggtcctagttgggtacattaccaatgtatgctgagagtactcgattcttttggtactgaacctgaatttaaccacgctaactatgctcaaagtaaaggtcataagacaccatggggaaagtggaatcttaacccccaacaattctacacaatgtttcctcacactccggataatagtttccttggctttgttgttgaacagcaccttaattcctctgacatacaccacattaatgaaattaagaggcagaaccaaagcctcgtttatggaaaagttgactccttttggaagaacaagaagatctatctggatataattcatacgtacatggaagttcatgctactgtttacgggtcatctaccaagaatattccctcctatgtcaagaatcatggtatactttctggaagagacttgcagttcttgcttagagaaaccaaactttttgtcggtcttggttttccttatgaaggtcctgcaccacttgaagctattgcaaatggatgtgcttttctcaacccaaagttcaatcctccaaagtcaagcaagaacaccgatttctttattggcaagcctacactaagagaacttacttcacaacacccttatgccgaagtttttattggaagaccacatgtttggacagtcgatttgaacaatcaagaggaagttgaggatgcagttaaggctatccttaaccagaagattgaaccatacatgccctacgaatttacatgtgaaggtatgttgcagcggattaacgcattcattgaaaagcaggacttttgtcatggtcaagttatgtggccacctctttcagctcttcaagtaaagttagcagaacctggtcaatcttgtaagcaagtttgtcaagagtcacagcttatttgcgaaccttcttttttccagcacctcaataaggataaggatatgctcaagtacaaggttacttgccaatcttctgaacttgcaaaggatatactcgttccatcttttgacccaaagaataagcattgcgtcttccaaggtgatcttctattgtttagttgtgctggagcacatcctagacatcaaagagtatgtccatgcagagatttcataaagggtcaagtagctctatgcaaggattgcctttga

SEQ ID NO: 22 - protein, 764 aa, Artificial
  Synthetic Construct
MGVFSNLRGPRAGATHDEFPATNGSPSSSSSPSSSIKRKLSNLLPLCVALVVIAEIGFLGRLPESSSMLREQILDLSKRYIKALAEENRNVVDGPYAGVMTAYDLKKTLAVLLDNILQRIGKLESKVDNLVVNGTGTNSTNSTTAVPSLVALEKINVADIINGAQEKCVLPPMDGYPHCEGKIKWMKDMWRSDPCYADYGVDGSTCSFFIYLSEVENWCPHLPWRAKNPYEEADHNSLAEIRTDFNILYSMMKKHEEFRWMRLRIRRMADAWIQAIKSLAEKQNLEKRKRKKVLVHLGLLTKESGFKIAETAFSGGPLGELVQWSDLITSLYLLGHDIRISASLAELKEIMKKVVGNRSGCPTVGDRIVELIYIDIVGLAQFKKTLGPSWVHYQCMLRVLDSFGTEPEFNHANYAQSKGHKTPWGKWNLNPQQFYTMFPHTPDNSFLGFVVEQHLNSSDIHHINEIKRQNQSLVYGKVDSFWKNKKIYLDIIHTYMEVHATVYGSSTKNIPSYVKNHGILSGRDLQFLLRETKLFVGLGFPYEGPAPLEAIANGCAFLNPKFNPPKSSKNTDFFIGKPTLRELTSQHPYAEVFIGRPHVWTVDLNNQEEVEDAVKAILNQKIEPYMPYEFTCEGMLQRINAFIEKQDFCHGQVMWPPLSALQVKLAEPGQSCKQVCQESQLICEPSFFQHLNKDKDMLKYKVTCQSSELAKDILVPSFDPKNKHCVFQGDLLLFSCAGAHPRHQRVCPCRDFIKGQVALCKDCL

SEQ ID NO: 23 - DNA, 609 bp, Artificial
  nucleotide sequence of aranesp (human erythropoietin), including secretion signal and HIS tag - codon optimized for Nicotiana benthamiana
atggcgaacaaacacttgtccctctccctcttcctcgtcctccttggcctgtcggccagcttggcctcaggtgcacctcctcgtctgatttgcgattctcgcgttttggaacggtacttactggaagctaaggaggctgagaatataacaacgggctgtaatgagacttgctccctaaatgagaatataactgtccccgatacaaaagtaaatttctacgcttggaagcgaatggaagttggacagcaagctgtggaagtgtggcaaggattggcattgcttagcgaagctgtcttaaggggacaagcactgctcgtaaatagtagtcaagttaacgagacacttcagctgcacgttgacaaggctgtttctggacttcgttctctaaccacactattgagggcactcggggctcagaaagaagctatttcaccgcctgacgccgcatctgcagctccacttcgaactatcactgctgatacctttagaaagctctttagggtgtattcaaactttcttagagggaagcttaaactctatacgggagaggcctgtagaactggtgatcgtttagttccaagaggctcacatcatcatcatcatcactag

SEQ ID NO: 24 - protein, 202 aa, Artificial
  Synthetic Construct
MANKHLSLSLFLVLLGLSASLASGAPPRLICDSRVLERYLLEAKEAENITTGCNETCSLNENITVPDTKVNFYAWKRMEVGQQAVEVWQGLALLSEAVLRGQALLVNSSQVNETLQLHVDKAVSGLRSLTTLLRALGAQKEAISPPDAASAAPLRTITADTFRKLFRVYSNFLRGKLKLYTGEACRTGDRLVPRGSHHHHHH

SEQ ID NO: 25 - DNA, 33 bp, artificial
  oligonucleotide
caccggtctcaaatgagtaaacggaatccgaag

SEQ ID NO: 26 - DNA, 36 bp, artificial
  oligonucleotide
caccggtctcatacccgatgagtgaaaaacgaagta

SEQ ID NO: 27 - DNA, 34 bp, artificial
  oligonucleotide
caccggtctcaaatgggtgttttctcgaatcttc

SEQ ID NO: 28 - DNA, 35 bp, artificial
  oligonucleotide
caatggtctcataccgagccgacccagaaacccga

SEQ ID NO: 29 - DNA, 39 bp, artificial
  oligonucleotide
caccggtctcaaggtcaaaatgggaaagaaaaactgatt

SEQ ID NO: 30 - DNA, 39 bp, artificial
  oligonucleotide
caccggtctcaaagctcagttggtggcttttttaatatg

SEQ ID NO: 31 - DNA, 36 bp, artificial
  oligonucleotide
caacggtctcaaggtgacgttgtggacgtttaccag

SEQ ID NO: 32 - DNA, 36 bp, artificial
  oligonucleotide
caccggtctcaaagcttagtcggcctttttcaggaa

SEQ ID NO: 33 - DNA, 36 bp, artificial
  oligonucleotide
caacggtctcaaggtcctgagtcatcttctatgctc

SEQ ID NO: 34 - DNA, 36 bp, artificial
  oligonucleotide
caacggtctcaaagctcagaggcaatccttacagag

SEQ ID NO: 35 - DNA, 19 bp, artificial
  primer
tcttgctgtggttgccctc

SEQ ID NO: 36 - DNA, 20 bp, artificial
  Reverse primer
ccaggaagccacgtctctga

SEQ ID NO: 37 - DNA, 19 bp, artificial
  epsps probe
ttgccgatggcccgacagc

SEQ ID NO: 38 - DNA, 20 bp, artificial
  forward primer Xylg19b
gcctctctgcccttttggat

SEQ ID NO: 39 - DNA, 26 bp, artificial
  Reverse primer XylTg19b
aaaggcatttactcgaattacaacaa

SEQ ID NO: 40 - DNA, 27 bp, artificial
  probe XylTg19b
tacgtgtaccatccccagaccccactc

SEQ ID NO: 41 - DNA, 24 bp, artificial
  Forward primer EF1alfa
gctgactgtgctgtcctgattatt

SEQ ID NO: 42 - DNA, 21 bp, artificial
  Reverse primer EF1alfa
tcacgggtctgtccatcctta

SEQ ID NO: 43 - DNA, 22 bp, artificial
  forward GnTIV target primer
acaagcctgtgaatgttgagag

SEQ ID NO: 44 - DNA, 22 bp, artificial
  Reverse GnT-IV target primer
cacctggatgttcttgattacc

SEQ ID NO: 45 - DNA, 22 bp, artificial
  Forward GnT-IVb target primer
ccaacagttttccatcatcttc

SEQ ID NO: 46 - DNA, 22 bp, artificial
  Reverse GnT-IVb target primer
actctaacagcaggttgcaatg

SEQ ID NO: 47 - DNA, 20 bp, artificial
  Forward GnT-Va target primer
tgcaccacttgaagctattg

SEQ ID NO: 48 - DNA, 20 bp, artificial
  Reverse GnT-Va target primer
aatcggtgttcttgcttgac

(19) United States
(12) Patent Application Publication
(10) Pub. No.: US 2012/0283420 A1
(43) Pub. Date: Nov. 8, 2012

(54) Production of Multi-Antennary N-Glycan Structures in Plants

(76) Inventors: Bieke Nagels, Grembergen (BE); Koen Weterings, Raleigh, NC (US)

(21) Appl. No.: 13/265,358

(22) PCT Filed: Apr. 19, 2010
(86) PCT No.: PCT/EP2010/002487

§ 371 Date: Jul. 30, 2012

Related U.S. Application Data
(60) Provisional application No. 61/171,594, filed on Apr. 22, 2009.

(30) Foreign Application Priority Data
Apr. 22, 2009 (EP) 09005631.8

(51) Int. Cl. C12P 21/00; C12N 5/10; A01H 5/00; C07K 14/00

(52) U.S. Cl. 530/395; 435/69.1; 435/419; 800/298

(57) ABSTRACT

The invention provides methods for producing multi-antennary glycoproteins in plant and plant cells. In particular the invention provides plants comprising a chimeric gene comprising glucosaminyltransferase IV and plants comprising two chimeric genes comprising glucosaminyltransferase IV and V.

### FIELD OF THE INVENTION

The current invention relates to the field of molecular farming, i.e. the use of plants and plant cells as bioreactors to produce biopharmaceuticals, particularly polypeptides and proteins with pharmaceutical interest such as therapeutic proteins, which have an N-glycosylation pattern that resembles mammalian glycosylation, in particular multi-antennary N-glycan structures. The invention may also be applied to alter the glycosylation pattern of proteins in plants for any purpose, including modulating the activity or half-life of endogenous plant proteins or proteins introduced in plant cells.

### BACKGROUND

Glycosylation is the covalent linkage of an oligosaccharide chain to a protein resulting in a glycoprotein. In many glycoproteins, the oligosaccharide chain is attached to the amide nitrogen of an asparagine (Asn) residue and leads to N-glycosylation. Glycosylation represents the most widespread post-translational modification found in natural and biopharmaceutical proteins. For example, more than half of the human proteins are glycosylated and their function frequently depends on particular glycoforms (glycans), which can affect their plasma half life, tissue targeting or even their biological activity. Similarly, more than one-third of approved biopharmaceuticals are glycoproteins and both their function and efficiency are affected by the presence and composition of their N-glycans. The functional activity of therapeutic glycoproteins is also frequently dependent on their glycosylation; this is the case, for example in blood factors, antibodies and interferons. This absolute requirement for glycosylation explains why many biopharmaceuticals are produced in expression systems with N-glycosylation capability. In recent years plants have emerged as an attractive system for the production of therapeutic proteins, as plants are generally considered to have several advantages, including the lack of animal pathogens such as prions and viruses, low cost and the large-scale production of safe and biologically active valuable recombinant proteins, the case of scale-up, efficient harvesting and storage possibilities. However, N-linked glycans from plants differ in many aspects from those of mammalian cells. In plants, beta(1,2)-xylose and alfa(1,3)-fucose residues have been shown to be linked to the core Man3GlucNAc2-Asn of glycans, whereas they are not detected on mammalian glycans, where sialic acid residues and terminal beta(1,4)-galactosyl structures occur instead. Another important difference between mammalian- and plant N-glycan structures is that plants do not synthesize multi-antennary glycans whereas it is calculated that about 10% of mammalian N-glycans are found to be of the tri- or tetra-antennary type. The latter type of multi-antennary glycans often determines the bio-availability and the half-life of glycoproteins. Thus, the commercial production of biotherapeutic glycoproteins of human origin in plants is currently hampered due to important differences in the N-glycosylation patterns between plants and humans. It is therefore envisaged that the administration of plant-made pharmaceutical glycoproteins to humans could lead to immunogenic or allergic reactions. Glyco-engineering with the combined knock-out/knock-in approach of glycosylation-related enzyme genes has been recognized for the avoidance of plant-specific glycan residues as well as the introduction of human glycosylation machinery in plants. Multi-antennary N-glycan structures, in particular tri- and/or tetra-antennary N-glycan structures, are not made in plants because plants not only lack GnT-IV and GnT-V activity (i.e. the enzymes involved in the formation of multi-antennary structures) but are also completely devoid of these GnT-IV and GnT-V sequences (for an overview of the glycosylation in several production systems such as plants see Jenkins et al (1996) Nature Biotechnology 14:975-979). The prior art does not describe plants that are capable of producing multi-antennary N-glycan structures. The mere introduction and overexpression of particular glucosaminyltransferases in production cell lines lacking said particular enzymes is not an obvious modification because toxicity has often been observed associated with the expression of an alien glucosaminyltransferase in an expression system. Indeed, the expression of glucosaminyltransferase-III in CHO cells resulted in growth inhibition due to cellular toxicity (Stanley P and Campbell C A (1984) Journal of Biological Chemistry 261:13370-13378) and the overexpression of glucosaminyltransferase V, a glycosyltransferase that produces tri-antennary sugar chains, also proved to be toxic (Umana et al (1999) Nature Biotechnology 17: 176-180).

The current invention provides methods and means to produce multi-antennary N-glycosylation structures of glycoproteins in plants and plant cells as will become apparent from the following description, examples, drawings and claims provided herein.

### SUMMARY OF THE INVENTION

It is one object of the invention to provide a method to produce multi-antennary glycoproteins (i.e. multi-antennary N-glycan structures) in plants or plant cells, said method comprising the steps of providing a plant cell with a chimeric gene comprising a plant-expressible promoter, a DNA region encoding a functional N-acetylglucosaminytransferase IV and a DNA region involved in transcription, termination and polyadenylation.

It is another object to provide a method to produce multi-antennary glycoproteins in plants and plant cells, said method comprising the steps of providing a plant cell with a first chimeric gene comprising a plant-expressible promoter, a DNA region encoding a functional N-acetylglucosaminytransferase IV and a DNA region involved in transcription, termination and polyadenylation and a second chimeric gene comprising a plant-expressible promoter, a DNA region encoding a functional N-acetylglucosaminytransferase V and a DNA region involved in transcription, termination and polyadenylation.

It is another object to provide a method for the production of multi-antennary glycoproteins in plants or plant cells wherein said plant or plant cells have a reduced level of beta(1,2)xylosyltransferase and alfa(1,3)fucosyltransferase activity, preferably no detectable beta(1,2)xylosyltransferase and no detectable alfa(1,3)fucosyltransferase activity.

In a particular embodiment said N-acetylglucosaminyltransferase IV and/or said N-acetylglucosaminyltransferase V are of the mammalian type. In another particular embodiment said N-acetylglucosaminyltransferase IV and/or said N-acetylglucosaminyltransferase V are of the hybrid type.

In another particular embodiment a third chimeric gene comprising a plant-expressible promoter, a DNA region encoding a functional beta(1,4)galactosyltransferase and a DNA region involved in transcription, termination and polyadenylation is expressed in a plant or plant cell capable of producing multi-antennary glycoproteins. In yet another particular embodiment a heterologous glycoprotein comprising a plant expressible promoter and a DNA region encoding said heterologous glycoprotein is additionally expressed in said plant or plant cells, said plant cells capable of producing multi-antennary N-glycans on glycoproteins.

It is another object of the invention to provide a multi-antennary glycoprotein produced in plant or plant cells wherein said plant or plant cells comprise 1) a chimeric gene comprising a plant-expressible promoter, a DNA region encoding a functional N-acetylglucosaminytransferase IV and a DNA region involved in transcription, termination and polyadenylation or 2) wherein said plant or plant cells comprise a first chimeric gene comprising a plant-expressible promoter, a DNA region encoding a functional N-acetylglucosaminytransferase IV and a DNA region involved in transcription, termination and polyadenylation and a second chimeric gene comprising a plant-expressible promoter, a DNA region encoding a functional N-acetylglucosaminytransferase V and a DNA region involved in transcription, termination and polyadenylation or 3) wherein said plant or plant cells comprise a first chimeric gene comprising a plant-expressible promoter, a DNA region encoding a functional N-acetylglucosaminytransferase IV and a DNA region involved in transcription, termination and polyadenylation and a second chimeric gene comprising a plant-expressible promoter, a DNA region encoding a functional N-acetylglucosaminytransferase V and a DNA region involved in transcription, termination and polyadenylation and a third chimeric gene comprising a plant-expressible promoter, a DNA region encoding a functional beta (1,4)-galactosyltransferase and a DNA region involved in transcription, termination and polyadenylation. In a particular embodiment said multi-antennary glycoprotein is a heterologous glycoprotein. In another particular embodiment said heterologous glycoprotein is produced in a plant or plant cell with a reduced level of beta(1,2)xylosyltransferase and alfa(1,3)fucosyltransferase activity, preferably no detectable beta(1,2)xylosyltransferase and no detectable alfa(1,3)fucosyltransferase activity. It is another object of the invention to provide a plant cell comprising a 1) a chimeric gene comprising a plant-expressible promoter, a DNA region encoding a functional N-acetylglucosaminytransferase IV and a DNA region involved in transcription, termination and polyadenylation or 2) a plant cell wherein said plant cell comprises a first chimeric gene comprising a plant-expressible promoter, a DNA region encoding a functional N-acetylglucosaminytransferase IV and a DNA region involved in transcription, termination and polyadenylation and a second chimeric gene comprising a plant-expressible promoter, a DNA region encoding a functional N-acetylglucosaminytransferase V and a DNA region involved in transcription, termination and polyadenylation or 3) a plant cell wherein said plant cell comprises a first chimeric gene comprising a plant-expressible promoter, a DNA region encoding a functional N-acetylglucosaminytransferase IV and a DNA region involved in transcription, termination and polyadenylation and a second chimeric gene comprising a plant-expressible promoter, a DNA region encoding a functional N-acetylglucosaminytransferase V and a DNA region involved in transcription, termination and polyadenylation and a third chimeric gene comprising a plant-expressible promoter, a DNA region encoding a functional beta (1,4)-galactosyltransferase and a DNA region involved in transcription, termination and polyadenylation. In a particular embodiment said plant cell further comprises a heterologous glycoprotein comprising the following operably linked nucleic acid molecules: i) a plant-expressible promoter, ii) a DNA region encoding a foreign protein and iii) a DNA region involved in transcription termination and polyadenylation. In a particular embodiment said plant cell comprises an N-acetylglucosaminyltransferase IV and/or V of the mammalian type. In another particular embodiment said plant cell comprises an N-acetylglucosaminyltransferase IV and/or V of the hybrid type. It is another object of the invention to provide a plant consisting essentially of the plant cells according to the before described objects and embodiments.

### BRIEF DESCRIPTION OF THE FIGURES

FIG. 1: MALDI-TOF MS analysis of endogenous glycosylated proteins in a xylosyltransferase negative and fucosyltransferase negative (XylT/FucT RNAi) background of Nicotiana benthamiana. 6 different hybrid N-acetylglucosaminyltransferases were transiently expressed in N. benthamiana as outlined in the examples. Four hybrid combinations (xylGnT-IVa, fucGnT-IVa, xylGnT-IVb and fucGnT-IVb) clearly show the production of tri-antennary structures in plant cell extracts (depicted as Gn[GnGn] as being glycans with the GlcNAcβ1-2Manα1-6(GlcNAcβ1-2(GlcNAcβ1-4)Manα1-3)Manβ1-4GlcNAcβ1-4GlcNAc-Asn conformation. In addition two hybrid combinations (xylGnT-Va and fucGnT-Va) also clearly show the formation of tri-antennary structures in plant cell extracts (depicted as [GnGn]Gn as being glycans with the GlcNAcβ1-6(GlcNAcβ1-2)Manα1-6(GlcNAcβ1-2Manα1-3)Manβ1-4GlcNAcβ1-4GlcNAc-Asn conformation. Please note that [GnGn]GnF is a product of GnT-Va but is also a fucosylated tri-antennary glycan due to remnant expression of fucosyltransferase in the XylT/FucT RNAi background and Gn[GnGn]F is a product of GnT-IVa or GnT-IVb but is also a fucosylated tri-antennary glycan due to remnant expression of fucosyltransferase in the XylT/FucT RNAi background on endogenous proteins.

FIG. 2: MALDI-TOF MS analysis of endogenous glycosylated proteins in a wild type background of Nicotiana benthamiana. 6 different hybrid N-acetylglucosaminyltransferases were transiently expressed in N. benthamiana as outlined in the examples. Three hybrid combinations (xylGnT-IVa, fucGnT-IVa and xylGnT-IVb) clearly show the production of tri-antennary structures in plant cell extracts (depicted as Gn[GnGn] as being glycans with the GlcNAcβ1-2Manα1-6(GlcNAcβ1-2(GlcNAcβ1-4)Manα1-3)Manβ1-4GlcNAcβ1-4GlcNAc-Asn conformation. In addition two hybrid combinations (xylGnT-Va and fucGnT-Va) also clearly show the formation of tri-antennary structures in plant cell extracts (depicted as [GnGn]Gn as being glycans with the GlcNAcβ1-6(GlcNAcβ1-2)Manα1-6(GlcNAcβ1-2Manα1-3)Manβ1-4GlcNAcβ1-4GlcNAc-Asn conformation. Please note that Gn[GnGn]F and Gn[GnGn]XF are products of GnT-IV but in addition are also fucosylated (F) or xylosylated and fucosylated (XF) structures. [GnGn]GnF and [GnGn]GnXF are products of GnT-Va but are also fucosylated (F) or fucosylated and xylosylated (XF).

FIG. 3: LC-ESI-MS analysis of N-glycans on endogenous proteins of wild type Nicotiana benthamiana plants. Since MALDI-TOF MS analysis is unable to distinguish between the tri-antennary N-glycans derived from GnT-IV or GnT-V, LC-ESI MS was performed. The data show the difference between the linkage of the introduced GlcNAc by the difference in elution time for samples of GnT-IV infiltration in comparison with GnT-V infiltration. Upon GnT-IV expression the tri-antennary glycans (GnGnGn-glycans) were GlcNAcβ1-2Manα1-6(GlcNAcβ1-2(GlcNAcβ1-4)Manα1-3)Manβ1-4GlcNAcβ1-4GlcNAc-Asn. Upon GnT-V expression the tri-antennary glycans (GnGnGn-glycans) were GlcNAcβ1-6(GlcNAcβ1-2)Manα1-6(GlcNAcβ1-2Manα1-3)Manβ1-4GlcNAcβ1-4GlcNAc-Asn.

Results are shown for 7 samples:
WT: non-infiltrated WT leaf sample
WT1: WT infiltrated with xylGnT-IVa
WT2: WT infiltrated with fucGnT-IVa
WT3: WT infiltrated with xylGnT-IVb
WT4: WT infiltrated with fucGnT-IVb
WT5: WT infiltrated with xylGnT-Va
WT6: WT infiltrated with fucGnT-Va
   For each sample the N-glycans were subjected to liquid chromatography which separates the different N-glycans based on conformation, size and hydrophobicity. Subsequently the N-glycans that eluted at a specific time point were subjected to mass spectrometry. The figure shows two profiles for each sample. Based on retention times of reference glycans, for each sample different traces for specific ions can be made. Both the upper and lower pattern of each sample are such selected ion traces that were subsequently subjected to mass spectrometry. In the upper pattern bi-antennary ions were selected, while in the lower pattern tri-antennary ions were selected. Samples WT 1 to WT 4 show an intense peak at the same retention time in the lower pattern which represents the presence of Gn[GnGn]-glycans. For sample WT 5 and WT 6, the peak in the lower pattern eluted at another time, which represent the difference in conformation of the third GlcNAc residue in as compared to WT 1-4 samples. WT 5 and WT 6 samples bear [GnGn]Gn-glycans.

FIG. 4: LC-ESI-MS analysis of N-glycans on endogenous proteins of XylT/FucT RNAi infiltrated leaf samples of Nicotiana benthamiana. Data is shown for three hybrid N-acetylglucosaminetransferase constructs infiltrated in the RNAi background. The data show the difference between the linkage of the introduced GlcNAc by the difference in elution time for samples of GnT-IV infiltration in comparison with GnT-V infiltration. Upon GnT-IV expression the tri-antennary glycans (GnGnGn-glycans) were GlcNAcβ1-2Manα1-6(GlcNAcβ1-2(GlcNAcβ1-4)Manα1-3)Manβ1-4GlcNAcβ1-4GlcNAc-Asn. Upon GnT-V expression the tri-antennary glycans (GnGnGn-glycans) were GlcNAcβ1-6(GlcNAcβ1-2)Manα1-6(GlcNAcβ1-2Manα1-3)Manβ1-4GlcNAcβ1-4GlcNAc-Asn. Panel 1-1 is a hybrid xylGnT-IVa, Panel 1-3 is a hybrid xylGnT-IVb and Panel 1-5 is a hybrid xylGnT-Va. For each sample the N-glycans were subjected to liquid chromatography which separates the different N-glycans based on conformation, size and hydrophobicity. Subsequently the N-glycans that eluted at a specific time point were subjected to mass spectrometry. The figure shows two profiles for each sample. Based on retention times of reference glycans for each sample different traces for specific ions can be made. Both the upper and lower pattern of each sample are such selected ion traces that were subsequently subjected to mass spectrometry. In the upper pattern bi-antennary ions were selected, while in the lower pattern tri-antennary ions were selected. Samples 1-1 and 1-3 show an intense peak at the same retention time in the lower pattern which represents the presence of Gn[GnGn]-glycans. For sample 1-5, the peak in the lower pattern eluted at another time, which represent the difference in conformation of the third GlcNAc residue in as compared to samples 1-1 and 1-3. Sample 1-5 carries [GnGn]Gn-glycans. For sample 1-1 also a mass spectrometry spectrum has been made showing clearly the presence of the most abundant N-glycans: GlcNAcβ1-2Manα1-6(GlcNAcβ1-2(GlcNAcβ1-4)Manα1-3)Manβ1-4GlcNAcβ1-4GlcNAc-Asn, GlcNAcβ1-2Manα1-6(GlcNAcβ1-2Manα1-3)Manβ1-4GlcNAcβ1-4GlcNAc-Asn, Manα1-6(Manα1-3)Manβ1-4GlcNAcβ1-4GlcNAc-Asn.

FIG. 5: MALDI-TOF MS analysis of endogenous glycosylated proteins of samples xylGnT-IVa RNAi-9, xylGnT-IVa RNAi-24, fucGnT-IVa RNAi-3, fucGnT-IVa RNAi-6, xylGnT-IVb RNAi-6 and xylGnT-IVb RNAi-20. All samples clearly show the production of tri-antennary N-glycan structures in plant cell extracts, depicted as Gn[GnGn], being glycans with the GlcNAcβ1-2Manα1-6(GlcNAcβ1-2(GlcNAcβ1-4)Manα1-3)Manβ1-4GlcNAcβ1-4GlcNAc-Asn conformation and [GnGn]Gn as being glycans with the GlcNAcβ1-6(GlcNAcβ1-2)Manα1-6(GlcNAcβ1-2Manα1-3)Manβ1-4GlcNAcβ1-4GlcNAc-Asn conformation. Please note that Gn[GnGn]F or [GnGn]GnF is also a product of GnT-IV and GnT-Va respectively but is also a fucosylated tri-antennary glycan due to remnant expression of fucosyltransferase in the XylT/FucT RNAi background and Gn[GnGn]F is a product of GnT-IVa or GnT-IVb but is also a fucosylated tri-antennary glycan due to remnant expression of fucosyltransferase in the XylT/FucT RNAi background on endogenous proteins.

FIG. 6: LC-ESI-MS analysis of N-glycans on endogenous proteins of stably transformers XylT/FucT RNAi and WT leaf samples of Nicotiana benthamiana. Data is shown for xylGnT-IVa RNAi-9 and -24, fucGnT-IVa RNAi-3, xylGnT-IVb RNAi-20, xylGnT-Va RNAi-7 and xylGnT-IVa WT-18. The data show the difference between the linkage of the introduced GlcNAc by the difference in elution time for samples of GnT-IV infiltration in comparison with GnT-V infiltration. Upon GnT-IV expression the tri-antennary glycans (GnGnGn-glycans) were GlcNAcβ1-2Manα1-6(GlcNAcβ1-2(GlcNAcβ1-4)Manα1-3)Manβ1-4GlcNAcβ1-4GlcNAc-Asn. Upon GnT-V expression the tri-antennary glycans (GnGnGn-glycans) were GlcNAcβ1-6(GlcNAcβ1-2)Manα1-6(GlcNAcβ1-2Manα1-3)Manβ1-4GlcNAcβ1-4GlcNAc-Asn. For each sample the N-glycans were subjected to liquid chromatography which separates the different N-glycans based on conformation, size and hydrophobicity. Subsequently the N-glycans that eluted at a specific time point were subjected to mass spectrometry. The figure shows four profiles for each sample. Based on retention times of reference glycans, for each sample different traces for specific ions can be made. All four patterns of each sample are such selected ion traces. In the upper pattern tri-antennary ions were selected, in the second bi-antennary N-glycans, in the third mono-antennary while in the lower pattern the trimannosylcore N-glycan ions were selected. Hybrid GnT-IV samples show an intense peak in the upper pattern which represents the presence of Gn[GnGn]-glycans, eluting a few minutes after the GnGn peak in the second pattern, while for hybrid GnT-V samples the tri-antennary N-glycan peak ([GnGn]Gn) elutes before the bi-antennary structure. This difference in elution time between the tri-antennary N-glycan peaks of hybrid GnT-IV and -V samples represents the difference in conformation of the third GlcNAc residue. Panel A: XylGnT-IVa RNAi-24 (Sample 48-2). Selected Ion Traces of masses 912.3435 (MM), 1115.423 (MGnisos), 1318.502 (GnGnisos) and 1521.582 (GnGnGnisos). Peaks marked with an X are empimers of natural Glycan Structures, Mass artefacts or small amounts of in source fragments. Panel B: XylGnT-IVa RNAi-9 (Sample 48-9) Selected Ion Traces of masses 912.3435 (MM), 1115.423 (MGnisos), 1318.502 (GnGnisos) and 1521.582 (GnGnGnisos). Peaks marked with an X are empimers of natural Glycan Structures, Mass artefacts or small amounts of in source fragments. Panel C: XylGnT-Va RNAi-7 (Sample 49-7). Selected Ion Traces of masses 912.3435 (MM), 1115.423 (MGnisos), 1318.502 (GnGnisos) and 1521.582 (GnGnGnisos). Peaks marked with an X are empimers of natural Glycan Structures, Mass artefacts or small amounts of in source fragments. Panel D: FucGnT-IVa RNAi-3 (Sample52-3). Selected Ion Traces of masses 912.3435 (MM), 1115.423 (MGnisos), 1318.502 (GnGnisos) and 1521.582 (GnGnGnisos). Peaks marked with an X are empimers of natural Glycan Structures, Mass artefacts or small amounts of in source fragments. Panel E: XylGnT-IVb RNAi-20 (Sample 54-20). Selected Ion Traces of masses 912.3435 (MM), 1115.423 (MGnisos), 1318.502 (GnGnisos) and 1521.582 (GnGnGnisos). Peaks marked with an X are empimers of natural Glycan Structures, Mass artefacts or small amounts of in source fragments. Panel F: RNAi background. Panel G: XylGnT-IVa WT-18.

FIG. 7: Chemiluminesce measured for a serial dilution of Neorecormon.

FIG. 8: In vitro activity of plant produced Aranesp. For each sample (WT=Aranesp expressed in WT plant, RNAi. Aranesp expressed in RNAi plant, 8=Aranesp expressed in xylGnT-Va RNAi plant, 10=Aranesp expressed in xylGnT-Va RNAi plant, 27=Aranesp expressed in fucGnT-IVa RNAi plant, 45=Aranesp expressed in fucGnT-IVa RNAi plant, 69=Aranesp expressed in fucGnT-IVa RNAi plant, 71=Aranesp expressed in fucGnT-IVa RNAi plant, 77=Aranesp expressed in xylGnT-IVb RNAi plant, 93=Aranesp expressed in xylGnT-IVb RNAi plant, 126=Aranesp expressed in xylGnT-IVa RNAi plant, 134=Aranesp expressed in xylGnT-IVa RNAi plant, 167=Aranesp expressed in xylGnT-IVa WT plant, 175=Aranesp expressed in xylGnT-IVa WT plant, 180=Aranesp expressed in fucGnT-IVa WT plant, 192=Aranesp expressed in fucGnT-IVa WT plant, 208=Aranesp expressed in xylGnT-Va WT plant, 210=Aranesp expressed in xylGnT-Va WT plant, the negative control (i.e. the untransformed WT plant) did not produce any chemiluminescence. The measured chemiluminescence is directly correlated with the activity (receptor binding) of Aranesp in the samples. The horizontal line represents the measured chemiluminescence after stimulation of the cells with 25 ng/ml Neorecormon.

### DETAILED DESCRIPTION OF DIFFERENT EMBODIMENT OF THE INVENTION

The current invention is based on the surprising observation that plant or plant cells comprising a chimeric gene comprising a glucosaminyltransferase IV are capable of producing multi-antennary N-glycans on glycoproteins which are expressed in said plant or plant cells and on the observation that plant and plant cells comprising two chimeric genes comprising a glucosyltransferase IV and V are capable of producing multi-antennary N-glycans on glycoproteins which are expressed in said plant or plant cells. The human N-acetylglucosaminyltransferase genes (GnT-IV en GnT-V), which are responsible for addition of N-acetylglucosamine residues on N-glycans in mammalian cells and thus contribute to the synthesis of multi-antennary N-glycan structures in mammalian cells, were introduced in wild type Arabidopsis thaliana and wild type Nicotiana benthamiana plants. In addition, these N-acetylglucosaminyltransferase genes were also introduced in partly humanized A. thaliana and N. benthamiana plants (i.e. by reducing the expression of (e.g. RNAi) or by knocking out the XylT and FucT genes these plants do not attach beta(1,2)-xylose and core-alfa(1,3)-fucose residues to their N-glycans and can be considered ‘partly humanized).

For both GnT-IV and GnT-V two genes are present in humans: GnT-IVa, GnT-IVb, GnT-Va and GnT-Vb (Taniguchi et al. (2002) Handbook of glycosyltransferases and related genes, Springer, Tokyo-Berlin-Heidelberg-New York-London, 670p., Kaneko et al. (2003) FEBS Letters 554, 515-519. In a preferred embodiment the N-acetylglucosaminyltransferases of the present invention are adapted to contain a different Golgi-localization signal. This is carried out by fusing the catalytic domains of the GnTs to the localization signals of plant enzymes which have their normal localization (or residence) in the Golgi. In a preferred way these hybrid GnT constructs are expressed in xylosyltransferase and fucosyltransferase (XylT/FucT) knock-out A. thaliana plants (Strasser et al. (2004) FEBS Letters 561, 132-136) and combined xylosyltransferase and fucosyltransferase RNAi down-regulated (XylT/FucT RNAi) N. benthamiana plants (Strasser et al. (2008) Plant Biotechnology Journal 6, 392-402).

In a first embodiment, the invention thus provides a method to produce multi-antennary glycoproteins in plants or plant cells comprising the steps of: a) providing a plant or plant cell with a chimeric gene comprising the following operably linked nucleic acid molecules: i) a plant-expressible promoter, ii) a DNA region encoding a functional N-acetylglucosaminyltransferase IV, iii) a DNA region involved in transcription termination and polyadenylation, and cultivating said plant or plant cell and isolating multi-antennary glycoproteins from said plant or plant cell.

In another embodiment the invention provides a method to produce multi-antennary glycoproteins in plants or plant cells comprising the steps of: a) providing a plant or plant cell with a first chimeric gene comprising the following operably linked nucleic acid molecules: i) a plant-expressible promoter, ii) a DNA region encoding a functional N-acetylglucosaminyltransferase IV, iii) a DNA region involved in transcription termination and polyadenylation, and a second chimeric gene comprising the following operably linked nucleic acid molecules: i) a plant-expressible promoter, ii) a DNA region encoding a functional N-acetylglucosaminyltransferase V, iii) a DNA region involved in transcription termination and polyadenylation, and cultivating said plant or plant cell and isolating multi-antennary glycoproteins from said plant or plant cell.

In yet another embodiment the methods to produce multi-antennary glycoproteins in plant or plant cells are carried out in plant or plant cells which have no detectable alfa-(1,3)xylosyltransferase and no detectable alfa-(1,3)fucosyltransferase activity. In a particular embodiment said N-acetylglucosaminyltransferase IV and/or said N-acetylglucosaminyltransferase V are of the mammalian type.

As used herein “a plant cell” is a cell of a higher plant belonging to the Angiospermae or the Gymospermae, but a plant cell can also be a lower plant cell such as plant cells belonging to Algae and Bryophyta. Preferably, the higher plant cell is a cell of a plant belonging to the Brassicaceae or the Solanaceae, including Arabidopsis or Nicotiana spp.

N-acetylglucosaminyltransferases (GnTs) belong to a class of glycosylation enzymes that modify N-linked oligosaccharides in the secretory pathway. These glycosyltransferases catalyze the transfer of a monosaccharide from specific sugar nucleotide donors onto a particular hydroxyl position of a monosaccharide in a growing glycan chain in one of two possible anomeric linkages (either alfa or beta). Specific GnTs add N-acetylglucosamine (GlcNAc) onto the mannose alfa 1,6 arm or the mannose alfa 1,3 arm of an N-glycan substrate (typically Man5GlcNAc2 which is designated as the mannose-5 core structure). The reaction product GlcNAcMan5GlcNAc2 is then be further modified into a bi-antennary structure in plants. The present invention shows that it is possible to further modify these bi-antennary structures into tri-antennary structures and even into tetra-antennary structures. Mammalian production systems are capable of producing tri-antennary N-glycan structures through the activity and the presence of GnT-IV or GnT-V. GnT-IV attaches a GlcNAc-residue in a beta(1,4)-binding to the terminal alfa(1,3)-mannose residue which is already substituted with a beta(1,2)-bound GlcNAc. GnT-V attaches a GlcNAc-residue in a beta(1,6)-binding to the terminal alfa(1,6)-mannose-residue which is already substituted with a beta(1,2)-bound GlcNAc. Tetra-antennary structures are produced by the combined enzymatic activities of both GnT-IV and GnT-V in the same mammalian cell.

N-acetylglucosaminyltransferase IV is the enzyme which characterizes the reaction between UDP-N-acetyl-D-glucosamine+3-(2-[N-acetyl-beta-D-glucosaminyl]-alpha-D-mannosyl)-beta-D-mannosyl-R=UDP+3-(2,4-bis[N-acetyl-beta-D-glucosaminyl]-alpha-D-mannosyl)-beta-D-mannosyl-R and wherein R represents the remainder of the N-linked oligosaccharide in the glycoprotein acceptor. The systematic name of this enzyme is UDP-N-acetyl-D-glucosamine:3-[2-(N-acetyl-beta-D-glucosaminyl)-alpha-D-mannosyl]-glycoprotein 4-beta-N-acetyl-D-glucosaminyltransferase (classification code EC2.4.1.145).

Alternative names are also alpha-1,3-mannosyl-glycoprotein 4-beta-N-acetylglucosaminyltransferase; N-acetylglucosaminyltransferase IV; N-glycosyl-oligosaccharide-glycoprotein N-acetylglucosaminyltransferase IV; beta-acetylglucosaminyltransferase IV; uridine diphosphoacetylglucosamine-glycopeptide beta4-acetylglucosaminyltransferase IV; alpha-1,3-mannosylglycoprotein beta-1,4-N-acetylglucosaminyltransferase and GnT-IV. For the purpose of the present invention we will use the terms N-acetylglucosaminyltransferase IV or GnT-IV.

N-acetylglucosaminyltransferase V is the enzyme which characterizes the reaction between UDP-N-acetyl-D-glucosamine+6-(2-[N-acetyl-beta-D-glucosaminyl]-alpha-D-mannosyl)-beta-D-mannosyl-R=UDP+6-(2,6-bis[N-acetyl-beta-D-glucosaminyl]-alpha-D-mannosyl)-beta-D-mannosyl-R and wherein R represents the remainder of the N-linked oligosaccharide in the glycoprotein acceptor. The systematic name of this enzyme is UDP-N-acetyl-D-glucosamine:6-[2-(N-acetyl-beta-D-glucosaminyl)-alpha-D-mannosyl]-glycoprotein 6-beta-N-acetyl-D-glucosaminyltransferase (classification code EC 2.4.1.155). Alternative names are also alpha-1,6-mannosyl-glycoprotein 6-beta-N-acetylglucosaminyltransferase; N-acetylglucosaminyltransferase V; alpha-mannoside beta-1,6-N-acetylglucosaminyltransferase; uridine diphosphoacetylglucosamine-alpha-mannoside

beta1->6-acetylglucosaminyltransferase; UDP-N-acetylglucosamine:alpha-mannoside-beta-1,6 N-acetylglucosaminyltransferase; alpha-1,3(6)-mannosylglycoprotein beta-1,6-N-acetylglucosaminyltransferase and GnT-V. For the purpose of the present invention we will use the terms N-acetylglucosaminyltransferase V or GnT-V.

Genes encoding GnT-IV (GnT-IVa or GnT-IVb) are well known and include the following database (National Centre for Biotechnology Information, NCBI) accession numbers identifying experimentally demonstrated and putative GnT-IV cDNA and gene sequences, parts thereof or homologous sequences: Homo Sapiens: NP_{—}055090 and NP_{—}036346, Pan troglodytes: XP_{—}001157522 and XP_{—}001151623, Macaca mulatta (rhesus monkey): XP_{—}001101794 and XP_{—}001102758, Mus musculus: NP_{—}666038 and NP_{—}776295, Rattus norvegicus: NP_{—}001121005 and NP_{—}001012225, Canis familiaris: XP_{—}531790 and XP_{—}538579, Bos taurus: NP_{—}803486, Monodelphis domestica (opossum): XP_{—}001371288, Gallus gallus: XP_{—}414605 and NP_{—}001012842, and Xenopus laevis (African clawed frog): NP_{—}001085444 Xenopus tropicalis (Western clawed frog): NP_{—}001096384, Danio rerio (zebrafish): NP_{—}001002180, NP_{—}001007438 and XP_{—}691496, Strongylocentrotus purpuratus (purple sea urchin): XP_{—}001190617, Nematostella vectensis (sea anemone): XP_{—}001632563 and Drosophila melanogaster (fruit fly): NP_{—}648721 and NP_{—}648720.

Genes encoding GnT-V are well known and include the following database (National Centre for Biotechnology Information, NCBI) accession numbers identifying experimentally demonstrated and putative GnT-V cDNA and gene sequences, parts thereof or homologous sequences: Homo sapiens: NP_{—}002401, Pan troglodytes: XP_{—}001151033, Mus musculus: NP_{—}660110, Rattus norvegicus: NP_{—}075583, Canis familiaris: XP_{—}541015, Bos taurus: XP_{—}001789652, Monodelphis domestica (opossum): XP_{—}001363544, Gallus gallus: XP_{—}422131, Danio rerio (zebrafish): NP_{—}001038776, Caenorhabditis elegans (nematode): NP_{—}491874.

The present invention provides methods for making a human-like glycoprotein in a plant or plant cell by introduction into said plant or plant cell of an N-acetylglucosaminyltransferase IV (GnT-IV) activity. In a preferred embodiment said GnT-IV activity is expressed in the plant or plant cell through the introduction of a chimeric gene comprising GnT-IV in said plant or plant cell. In a more preferred embodiment the expression of GnT-IV in said plant or plant cell leads to the production of N-glycans comprising tri-antennary glycoproteins in said plant or plant cells. Said tri-antennary structure is typically an N-glycan GlcNAc3Man3GlcNAc2-structure. In another embodiment the introduction into a plant or plant cell of an N-acetylglucosaminyltransferase V (GnT-V) activity leads to the production of a human-like glycoprotein in said plant or plant cell. In a preferred embodiment said GnT-V activity is expressed in the plant or plant cell through the introduction of a chimeric gene comprising GnT-V in said plant or plant cell. In more preferred embodiment the expression of GnT-V in said plant or plant cell leads to the production of N-glycans comprising tri-antennary glycoproteins in said plant or plant cells. Said tri-antennary N-glycan structure is a GlcNAc3Man3GlcNAc2-structure.

In another embodiment the invention provides methods for making a human-like glycoprotein in a plant or plant cell by combined introduction into said plant or plant cell of an N-acetylglucosaminyltransferase IV (GnT-IV) and an N-acetylglucosaminyltransferase V activity (GnT-V). In a preferred embodiment said combined GnT-IV and GnT-V activity is expressed in the plant or plant cell through the introduction of a chimeric gene comprising GnT-IV and GnT-V or through the introduction of two chimeric genes, one comprising GnT-IV and the other comprising GnT-V in said plant or plant cell. In a more preferred embodiment the combined expression of GnT-IV and GnT-V in said plant or plant cell leads to the production of N-glycans comprising tetra-antennary glycoproteins in said plant or plant cells. Said tetra-antennary N-glycan structure is a GlcNAc4Man3GlcNAc2-structure. In some embodiments, the resulting plant or plant cell includes an N-glycan that comprises both GlcNAc3Man3GlcNAc2- and GlcNAc4Man3GlcNAc2-structures.

The introduction of a chimeric gene comprising GnT-V in a plant or plant cells leads to the production of tri-antennary glycoproteins in said plant or plant cells. The introduction of a combination of a chimeric gene comprising GnT-IV and a chimeric gene comprising GnT-V in a plant or plant cells leads to the production of tetra-antennary glycoproteins in said plant or plant cells. In the present invention “multi-antennary glycoproteins” can be either tri-antennary glycoproteins or tetra-antennary glycoproteins or can be a combination (i.e. a mixture) of tri-antennary and tetra-antennary glycoproteins.

In a preferred embodiment said N-acetylglucosaminyltransferase IV and/or said N-acetylglucosaminyltransferase V which are expressed in said plant or plant cell are of the hybrid type. Glycosyltransferases such as GnT-IV and GnT-V have an N-terminal localization region which determines the localization of this enzyme in the ER or Golgi membrane. Said glycosyltransferases can be expressed in plants as they occur in for example mammals, but they can also be expressed as a hybrid protein between two (or part of two) different glycosyltransferases. In this case the localization is determined by one enzyme and the catalytic activity by a second enzyme. An example of such hybrid GnT-IV enzyme is a fusion between the localization signal (LS) of fucosyltransferase B and the catalytic domain of GnT-IVa such as provided in SEQ ID NO: 13. Thus in the latter hybrid enzyme the LS-region from the GnT-IVa is replaced with the LS region form another Golgi-localized protein (i.e. for example the LS region of the plant fucosyltransferase B). Such a Golgi localization signal is also designated as a cytoplasmic, transmembrane and stem region (CTS-region) in the art and can be easily recognized by a person skilled in the art. The resulting hybrid enzyme has GnT-IVa activity and the localization signal of the fucosyltransferase B. A non-limiting list of localization signals which can be used in the construction of hybrid N-acetylglucosaminyltransferases IV and V comprises the rat alfa(2,6)-sialyltransferase (Genbank accession M18769), a plant xylosyltransferase, a plant fucosyltransferase, an eukaryotic N-acetylglucosaminyltransferase I or II, a plant galactosyltransferase or an eukaryotic mannosidase I, II or III.

In yet another embodiment the expression of a functional N-acetylglucosaminyltransferase IV or a combined expression of a functional N-acetylglucosaminyltransferase IV and V is in a plant or plant cell which has a reduced expression of beta-(1,2)xylosyltransferase and a reduced expression of alfa-(1,3) fucosyltransferase. In a preferred embodiment said plant or plant cell has no detectable beta-(1,2)xylosyltransferase and no detectable alfa-(1,3)fucosyltransferase.

The level of beta(1,2)xylosyltransferase and alfa(1,3)fucosyltransferase activity can conveniently be reduced or eliminated by identifying plant cells having a null mutation in all of the genes encoding beta(1,2)xylosyltransferase and in all of the genes encoding alfa(1,3)fucosyltransferase.

Genes encoding alfa(1,3)fucosyltransferase (FucT) in plants are well known and include the following database entries identifying experimentally demonstrated and putative FucT cDNA and gene sequences, parts thereof or homologous sequences: NM 112815 (Arabidopsis thaliana), NM103858 (Arabidopsis thaliana), AJ 618932 (Physcomitrella patens) At1g49710(Arabidopsis thaliana) and At3g19280 (Arabidopsis thaliana). DQ789145 (Lemna minor), AY557602 (Medicago truncatula) Y18529 (Vigna radiata) AP004457 (Oryza sativa), AJ891040 encoding protein CA170373 (Populus alba x Populus tremula) AY082445 encoding protein AAL99371 (Medicago sativa) AJ582182 encoding protein CAE46649 (Triticum aestivum) AJ582181 encoding protein CAE46648 (Hordeum vulgare)(all sequences herein incorporated by reference).

Genes encoding beta(1,2)xylosyltransferase (XylT) in plants are well known and include the following database entries identifying experimentally demonstrated and putative XylT cDNA and gene sequences, parts thereof or homologous sequences: AJ627182, AJ627183 (Nicotiana tabacum cv. Xanthi), AM179855 (Solanum tuberosum), AM179856 (Vitis vinifera), AJ891042 (Populus alba x Populus tremula), AY302251 (Medicago sativa), AJ864704 (Saccharum officinarum), AM179857 (Zea mays), AM179853 (Hordeum vulgare), AM179854 (Sorghum bicolor), BD434535, AJ277603, AJ272121, AF272852, AX236965 (Arabidopsis thaliana), AJ621918 (Oryza sativa), AR359783, AR359782, AR123000, AR123001 (Soybean), AJ618933 (Physcomitrella patens) and At5g55500 (Arabidopsis thaliana) as well as the nucleotide sequences from Nicotiana species described in application PCT/EP2007/002322 (all sequences herein incorporated by reference).

Based on the available sequences, the skilled person can isolate genes encoding alfa(1,3)fucosyltransferase or genes encoding beta(1,2)xylosyltransferase from plants other than the plants mentioned above. Homologous nucleotide sequence may be identified and isolated by hybridization under stringent conditions using as probes identified nucleotide sequences.

“Stringent hybridization conditions” as used herein means that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C., preferably twice for about 10 minutes. Other hybridization and wash conditions are well known and are exemplified in Sambrook et al, Molecular Cloning: A Laboratory Manual, Second Edition, Cold Spring Harbor, N.Y. (1989), particularly chapter 11.

Nucleotide sequences obtained in this way should be verified for encoding a polypeptide having an amino acid sequence which is at least 80% to 95% identical to a known alfa(1,3)fucosyltransferase or beta(1,2)xylosyltransferase from plants.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J Mol Biol. 48: 443-453) The computer-assisted sequence alignment above, can be conveniently performed using standard software program such as GAP which is part of the Wisconsin Package Version 10.1 (Genetics Computer Group, Madision, Wis., USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical. It is clear than when RNA sequences are the to be essentially similar or have a certain degree of sequence identity with DNA sequences, thymine (T) in the DNA sequence is considered equal to uracil (U) in the RNA sequence.

Other sequences encoding alfa(1,3)fucosyltransferase or beta(1,2)xylosyltransferase may also be obtained by DNA amplification using oligonucleotides specific for genes encoding alfa(1,3)fucosyltransferase or beta(1,2)xylosyltransferase as primers, such as but not limited to oligonucleotides comprising about 20 to about 50 consecutive nucleotides from the known nucleotide sequences or their complement.

The art also provides for numerous methods to isolate and identify plant cells having a mutation in a particular gene.

Mutants having a deletion or other lesion in the alfa(1,3)fucosyltransferase or beta(1,2) xylosyltransferase encoding genes can conveniently be recognized using e.g. a method named “Targeting induced local lesions in genomes (TILLING)”. Plant Physiol. 2000 June; 123(2):439-42. Plant cells having a mutation in the desired gene may also be identified in other ways, e.g. through amplification and nucleotide sequence determination of the gene of interest. Null mutations may include e.g. genes with insertions in the coding region or gene with premature stop codons or mutations which interfere with the correct splicing. Mutants may be induced by treatment with ionizing radiation or by treatment with chemical mutagens such as EMS.

The level of beta(1,2)xylosyltransferase and alfa(1,3)fucosyltransferase activity can also conveniently be reduced or eliminated by transcriptional or post-transcriptional silencing of the expression of endogenous beta(1,2)xylosyltransferase and alfa(1,3) fucosyltransferase encoding genes. To this end a silencing RNA molecule is introduced in the plant cells targeting the endogenous beta(1,2)xylosyltransferase and alfa(1,3) fucosyltransferase encoding genes. As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region. hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small ranging in size from about 30 to about 42 bp, but not much longer than 94 by (see WO04/073390, herein incorporated by reference). Silencing RNA may also be artificial micro-RNA molecules as described e.g. in WO2005/052170, WO2005/047505 or US 2005/0144667 (all documents incorporated herein by reference)

In another embodiment, the silencing RNA molecules are provided to the plant cell or plant by producing a transgenic plant cell or plant comprising a chimeric gene capable of producing a silencing RNA molecule, particularly a double stranded RNA (“dsRNA”) molecule, wherein the complementary RNA strands of such a dsRNA molecule comprises a part of a nucleotide sequence encoding a XylT or FucT protein.

The plant or plant cells according to the invention also can further comprise a beta(1,4) galactosyltransferase activity. Conveniently, such activity may be introduced into plant cells by providing them with a chimeric gene comprising a plant-expressible promoter operably linked to a DNA region encoding a beta(1,4)galactosyltransferase and optionally a 3′ end region involving in transcription termination and polyadenylation functional in plant cells. The term “beta-(1,4)galactosyltransferase” refers to the glycosyltransferase designated as EC2.4.1.38 that is required for the biosynthesis of the backbone structure from type 2 chain (Galbeta1→4GlcNAc), which appears widely on N-linked glycans, i.e., which enzyme has galactosylating activity on N-linked glycans. Useful beta(1,4)galactosyltransferases are derived from human, mouse, rat as well as orthologs of beta(1,4)galactosyltransferase from non-mammalian species such as chicken and zebrafish (see also WO2008125972).

Regions encoding a beta(1,4)galactosyltransferase are preferably obtained from mammalian organisms, including humans, but may be obtained from other organisms as well. NMO22305 (Mus musculus) NM146045 (Mus musculus) NM 004776 (Homo sapiens) NM 001497(Homo sapiens) are a few database entries for genes encoding a β(1,4)galactosyltransferase. Others database entries for β(1,4)galactosyltransferases include AAB05218 (Gallus gallus), XP693272 (Danio rerio), CAF95423 (Tetraodon nigroviridis) or NP001016664 (Xenopus tropicalis) (all sequence herein incorporated by reference).

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Harpster et al. (1988) Mol Gen Genet. 212(1):182-90, the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8(1):15-30), stem-specific promoters (Keller et al., (1988) EMBO J. 7(12): 3625-3633), leaf specific promoters (Hudspeth et al. (1989) Plant Mol Biol. 12: 579-589), mesophyl-specific promoters (such as the light-inducible Rubisco promoters), root-specific promoters (Keller et al. (1989) Genes Dev. 3: 1639-1646), tuber-specific promoters (Keil et al. (1989) EMBO J. 8(5): 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like.

According to the invention, the N-glycan profile of glycoproteins may be altered or modified. The glycoproteins may be glycoproteins endogeneous to the cell of the higher plant, and may result in altered functionality, folding or half-life of these proteins. Glycoproteins also include proteins which are foreign to the cell of the higher plant (i.e. a heterologous glycoprotein), i.e. which are not normally expressed in such plant cells in nature. These may include mammalian or human proteins, which can be used as therapeutics such as e.g. monoclonal antibodies. Conveniently, the foreign glycoproteins may be expressed from chimeric genes comprising a plant-expressible promoter and the coding region of the glycoprotein of interest, whereby the chimeric gene is stably integrated in the genome of the plant cell. Methods to express foreign proteins in plant cells are well known in the art. Alternatively, the foreign glycoproteins may also be expressed in a transient manner, e.g. using the viral vectors and methods described in WO02/088369, WO2006/079546 and WO2006/012906 or using the viral vectors described in WO89/08145, WO93/03161 and WO96/40867 or WO96/12028.

In a particular embodiment the plant or plant cells of the invention produce at least 10%, at least 20%, at least 30%, at least 40%, at least 50%, at least 60%, at least 70%, at least 80%, at least 90% or even higher amounts of multi-antennary (i.e. tri- or tetra-antennary or even a mixture of tri- and tetra-antennary glycan structures) glycoprotein structures on the produced glycoprotein. The amount of multi-antennary glycan structures associated with a produced heterologous glycoprotein can be determined according to the methods described in this application and is usually expressed as a relative abundance of bi- and tri-antennary N-glycans as shown in Table 2. To determine the relative amounts of tri- and tetra-antennary N-glycans in a MALDI-TOF MS spectrum, first the heights of all peaks are summed. Than for each individual peak, N-glycan structure, the relative abundance can be calculated by dividing the height of the peak of interest by the total sum of all heights and multiplying this by 100.

By “heterologous protein” it is understood a protein (i.e. a polypeptide) that is not expressed by the plant or plant cells in nature. This is in contrast with a homologous protein which is a protein naturally expressed by a plant or plant cell. Heterologous and homologous polypeptides that undergo post-translational N-glycosylation are referred to herein as heterologous or homologous glycoproteins.

Examples of heterologous proteins of interest that can be advantageously produced by the methods of this invention include, without limitation, cytokines, cytokine receptors, growth factors (e.g. EGF, HER-2, FGF-alpha, FGF-beta, TGF-alpha, TGF-beta, PDGF, IGF-I, IGF-2, NGF), growth factor receptors. Other examples include growth hormones (e.g. human growth hormone, bovine growth hormone); insulin (e.g., insulin A chain and insulin B chain), pro-insulin, erythropoietin (EPO), colony stimulating factors (e.g. G-CSF, M-CSF); interleukins; vascular endothelial growth factor (VEGF) and its receptor (VEGF-R), interferons, tumor necrosis factor and its receptors, thrombopoietin (TPO), thrombin, brain natriuretic peptide (BNP); clotting factors (e.g. Factor VIII, Factor IX, von Willebrands factor and the like), anti-clotting factors; tissue plasminogen activator (TPA), urokinase, follicle stimulating hormone (FSH), luteinizing hormone (LH), calcitonin, CD proteins (e.g., CD2, CD3, CD4, CD5, CD7, CD8, CDI Ia, CDI Ib, CD18, CD19, CD20, CD25, CD33, CD44, CD45, CD71, etc.), CTLA proteins (e.g.CTLA4); T-cell and B-cell receptor proteins, bone morphogenic proteins (BNPs, e.g. BMP-I, BMP-2, BMP-3, etc.), neurotrophic factors, e.g. bone derived neurotrophic factor (BDNF), neurotrophins, e.g. rennin, rheumatoid factor, RANTES, albumin, relaxin, macrophage inhibitory protein (e.g. MIP-I, MIP-2), viral proteins or antigens, surface membrane proteins, on channel proteins, enzymes, regulatory proteins, immunomodulatory proteins, (e.g. HLA, MHC, the B7 family), homing receptors, transport proteins, superoxide dismutase (SOD), G-protein coupled receptor proteins (GPCRs), neuromodulatory proteins, Alzheimer's Disease associated proteins and peptides. Fusion proteins and polypeptides, chimeric proteins and polypeptides, as well as fragments or portions, or mutants, variants, or analogs of any of the aforementioned proteins and polypeptides are also included among the suitable proteins, polypeptides and peptides that can be produced by the methods of the present invention. In a preferred embodiment, the protein of interest is a glycoprotein. One class of glycoproteins are viral glycoproteins, in particular subunits, than can be used to produce for example a vaccine. Some examples of viral proteins comprise proteins from rhinovirus, poliomyelitis virus, herpes virus, bovine herpes virus, influenza virus, newcastle disease virus, respiratory syncitio virus, measles virus, retrovirus, such as human immunodeficiency virus or a parvovirus or a papovavirus, rotavirus or a coronavirus, such as transmissable gastroenteritisvirus or a flavivirus, such as tick-borne encephalitis virus or yellow fever virus, a togavirus, such as rubella virus or eastern, western, or venezuelean equine encephalomyelitis virus, a hepatitis causing virus, such as hepatitis A or hepatitis B virus, a pestivirus, such as hog cholera virus or a rhabdovirus, such as rabies virus. In another preferred embodiment, the heterologous glycoprotein is an antibody or a fragment thereof. The term “antibody” refers to recombinant antibodies (for example of the classes IgD, IgG, IgA, IgM, IgE) and recombinant antibodies such as single-chain antibodies, chimeric and humanized antibodies and multi-specific antibodies. The term “antibody” also refers to fragments and derivatives of all of the foregoing, and may further comprises any modified or derivatised variants thereof that retain the ability to specifically bind an epitope. Antibody derivatives may comprise a protein or chemical moiety conjugated to an antibody. A monoclonal antibody is capable of selectively binding to a target antigen or epitope. Antibodies include, monoclonal antibodies (mAbs), humanized or chimeric antibodies, camelized antibodies, camelid antibodies (Nanobodies®), single chain antibodies (scFvs), Fab fragments, F(ab′)₂ fragments, disulfide-linked Fvs (sdFv) fragments, anti-idiotypic (anti-Id) antibodies, intra-bodies, synthetic antibodies, and epitope-binding fragments of any of the above. The term “antibody” also refers to fusion protein that includes a region equivalent to the Fc region of an immunoglobulin. Also envisaged is the production in the plant or plant cells of the invention of so called dual-specificity antibodies (Bostrom J et al (2009) Science 323, 1610-1614).

Preferred antibodies within the scope of the present invention include those comprising the amino acid sequences of the following antibodies: anti-HER2 antibodies including antibodies comprising the heavy and light chain variable regions (see U.S. Pat. No. 5,725,856) or Trastuzumab such as HERCEPTIN™; anti-CD20 antibodies such as chimeric anti-CD20 as in U.S. Pat. No. 5,736,137, a chimeric or humanized variant of the 2H7 antibody as in U.S. Pat. No. 5,721,108; anti-VEGF antibodies including humanized and/or affinity matured anti-VEGF antibodies such as the humanized anti-VEGF antibody huA4.6.1 AVASTIN™ (WO 96/30046 and WO 98/45331); anti-EGFR (chimerized or humanized antibody as in WO 96/40210); anti-CD3 antibodies such as OKT3 (U.S. Pat. No. 4,515,893); anti-CD25 or anti-tac antibodies such as CHI-621 (SIMULECT) and (ZENAPAX) (U.S. Pat. No. 5,693,762). The present invention provides a method for the production of an antibody which comprises culturing a transformed plant cell or growing a transformed plant of the present invention. The produced antibody may be purified and formulated in accordance with standard procedures.

The nucleotide sequences of the glycosyltransferases and/or the heterologous genes may be codon optimized to increase the level of expression within the plant. By codon optimization it is meant the selection of appropriate DNA nucleotides for the synthesis of oligonucleotide building blocks, and their subsequent enzymatic assembly, of a structural gene or fragment thereof in order to approach codon usage in plants.

In certain embodiments methods for obtaining a desired glycoprotein or functional fragment thereof comprise cultivating a plant described herein until sad plant has reached a harvestable stage, harvesting and fractionating the plant to obtain fractionated plant material and at least partly isolating said glycoprotein from said fractionated plant material.

In certain embodiments methods for obtaining a desired glycoprotein or functional fragment thereof comprise growing recombinant plant cells in cell culture in a fermentor until sad cell culture has reached a harvestable stage or the desired glycoprotein can be collected from the medium. The glycoproteins described herein, such as e.g., antibodies, vaccines, cytokines and hormones, may be purified by standard techniques well known to those of skill in the art. Such recombinantly produced proteins may be directly expressed or expressed as a fusion protein. The recombinant protein is purified by a combination of cell lysis sonication, French press) and affinity chromatography or other affinity-based method. For fusion products, subsequent digestion of the fusion protein with an appropriate proteolytic enzyme releases the desired recombinant protein.

The proteins described herein, recombinant or synthetic, may be purified to substantial purity by standard techniques well known in the art, including detergent solubilization, selective precipitation with such substances as ammonium sulfate, column chromatography, immunopurification methods, and others. See, for instance, FR. Scopes, Protein Purification Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. The protein may then be isolated from cells expressing the protein and further purified by standard protein chemistry techniques as described herein. Detection of the expressed protein is achieved by methods known in the art and include, for example, radioimmunoassays, Western blotting techniques or immunoprecipitation.

In yet another embodiment the invention provides a multi-antennary glycoprotein, such as a heterologous glycoprotein, obtained by the methods described herein before. In a particular embodiment such a multi-antennary glycoprotein is a tri-antennary glycoprotein optionally carrying a beta-(1,2)xylose sugar, optionally carrying a beta-(1,2) xylose sugar and an alfa-(1,3)fucose sugar. In another particular embodiment such a multi-antennary glycoprotein is a tetra-antennary glycoprotein optionally carrying a beta-(1,2)xylose sugar, optionally carrying a beta-(1,2)xylose sugar and an alfa-(1,3)fucose sugar. In yet another embodiment said tri-antennary or tetra-antennary glycoprotein also comprises at least one beta-(1,4)-galactose sugar.

In yet another embodiment the invention provides a plant cell comprising a chimeric gene comprising the following operably linked nucleic acid molecules. i) a plant-expressible promoter, ii) a DNA region encoding a functional N-acetylglucosaminyltransferase IV, and iii) a DNA region involved in transcription termination and polyadenylation.

In yet another embodiment the invention provides a plant cell comprising a first chimeric gene comprising the following operably linked nucleic acid molecules: i) a plant-expressible promoter, ii) a DNA region encoding a functional N-acetylglucosaminyltransferase IV, and iii) a DNA region involved in transcription termination and polyadenylation and a second chimeric gene comprising the following operably linked nucleic acid molecules: i) a plant-expressible promoter, ii) a DNA region encoding a functional N-acetylglucosaminyltransferase V, and iii) a DNA region involved in transcription termination and polyadenylation.

In yet another embodiment the invention provides a plant cell comprising a first chimeric gene comprising the following operably linked nucleic acid molecules: i) a plant-expressible promoter, ii) a DNA region encoding a functional N-acetylglucosaminyltransferase IV, and iii) a DNA region involved in transcription termination and polyadenylation; a second chimeric gene comprising the following operably linked nucleic acid molecules: i) a plant-expressible promoter, ii) a DNA region encoding a functional N-acetylglucosaminyltransferase V, and iii) a DNA region involved in transcription termination and polyadenylation and a third chimeric gene comprising the following operably linked nucleic acid molecules: i) a plant-expressible promoter, ii) a DNA region encoding a functional beta(1,4)-galactosyltransferase and iii) a DNA region involved in transcription termination and polyadenylation.

In yet another embodiment the invention provides a plant cell comprising a first chimeric gene comprising the following operably linked nucleic acid molecules: i) a plant-expressible promoter, ii) a DNA region encoding a functional N-acetylglucosaminyltransferase IV, and iii) a DNA region involved in transcription termination and polyadenylation; a second chimeric gene comprising the following operably linked nucleic acid molecules: i) a plant-expressible promoter, ii) a DNA region encoding a functional N-acetylglucosaminyltransferase V, and iii) a DNA region involved in transcription termination and polyadenylation and a third chimeric gene comprising the following operably linked nucleic acid molecules: i) a plant-expressible promoter, ii) a DNA region encoding a heterologous glycoprotein and iii) a DNA region involved in transcription termination and polyadenylation. In a particular embodiment a plant cell comprises a fourth chimeric gene comprising the following operably linked nucleic acid molecules: i) a plant-expressible promoter, ii) a DNA region encoding a functional beta(1,4)-galactosyltransferase and iii) a DNA region involved in transcription termination and polyadenylation. In a preferred embodiment the plant cell wherein the chimeric genes are introduced has no detectable beta-(1,2)xylosyltransferase and no detectable alfa (1,3)fucosyltransferase activity.

In yet another particular embodiment the N-acetylglucosaminyltransferase IV and/or V genes are of the mammalian type and are optionally of the hybrid type.

In yet another embodiment the invention provides a plant essentially consisting of the recombinant plant cells herein above described.

The methods and means described herein are believed to be suitable for all plant cells and plants, gymnosperms and angiosperms, both dicotyledonous and monocotyledonous plant cells and plants including but not limited to Arabidopsis, alfalfa, barley, bean, corn or maize, cotton, flax, oat, pea, rape, rice, rye, safflower, sorghum, soybean, sunflower, tobacco and other Nicotiana species, including Nicotiana benthamiana, wheat, asparagus, beet, broccoli, cabbage, carrot, cauliflower, celery, cucumber, eggplant, lettuce, onion, oilseed rape, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon Brassica vegetables, sugarcane, vegetables (including chicory, lettuce, tomato) and sugarbeet.

Methods for the introduction of chimeric genes into plants are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc. The transformed cells obtained in this way may then be regenerated into mature fertile plants.

A DNA sequence encoding a heterologous protein or polypeptide can encode translation codons that reflect the preferred codon usage of a plant cell or plant. For example, if the host cell or organism species is Nicotiana benthamiana, a codon usage table such as that published on the internet at http://www.kazusa.or.jp/codon/cgi-bin/showcodon.cgi?species=4100 can be used to select codons or their complements in designing an artificial DNA sequence or modifying a naturally occurring DNA sequence. It is expected that use of preferred codons in a coding sequence will lead to higher efficiency of translation of a transgene (i.e. in the present case a heterologous protein, in particular a heterologous glycoprotein) in a transgenic plant cell or plant.

Gametes, seeds, embryos, progeny, hybrids of plants, or plant tissues including stems, leaves, stamen, ovaria, roots, meristems, flowers, seeds, fruits, fibers comprising the chimeric genes of the present invention, which are produced by traditional breeding methods are also included within the scope of the present invention.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined, may comprise additional DNA regions etc.

The following non-limiting Examples describe the introduction of GnT-IV activity, the combined introduction of GnT-IV activity and GnT-V activity in a wild type plant background or in a mutant plant background wherein said plants have a reduced or absent expression of beta(1,2)-xylosyltransferase and a reduced or absent expression of alfa(1,3)-fucosyltransferase. The examples convincingly show that tri-antennary and tetra-antennary N-glycans are synthesized on endogenous and heterologous glycoproteins in plants.

Unless stated otherwise in the Examples, all recombinant techniques are carried out according to standard protocols as described in “Sambrook J and Russell D W (eds.) (2001) Molecular Cloning: A Laboratory Manual, 3rd Edition, Cold Spring Harbor Laboratory Press, New York” and in “Ausubel F A, Brent R, Kingston R E, Moore D D, Seidman J G, Smith J A and Struhl K (eds.) (2006) Current Protocols in Molecular Biology. John Wiley & Sons, New York”. Standard materials and references are described in “Croy R D D (ed.) (1993) Plant Molecular Biology LabFax, BIOS Scientific Publishers Ltd., Oxford and Blackwell Scientific Publications, Oxford” and in “Brown T A, (1998) Molecular Biology LabFax, 2nd Edition, Academic Press, San Diego”. Standard materials and methods for polymerase chain reactions (PCR) can be found in “McPherson M J and Møller S G (2000) PCR (The Basics), BIOS Scientific Publishers Ltd., Oxford” and in “PCR Applications Manual, 3rd Edition (2006), Roche Diagnostics GmbH, Mannheim or www.roche-applied-science.com”

Throughout the description and Examples, reference is made to the following sequences:
SEQ ID NO 1: nucleotide sequence of localisation signal of xylosyltransferase of Arabidopsis thaliana
SEQ ID NO 2: amino acid sequence of SEQ ID NO: 1
SEQ ID NO 3: nucleotide sequence of localisation signal of fucosyltransferase B of Arabidopsis thaliana
SEQ ID NO 4: amino acid sequence of SEQ ID NO: 3
SEQ ID NO 5: nucleotide sequence catalytic domain of GnT-IVa from Homo sapiens
SEQ ID NO 6: amino acid sequence of SEQ ID NO: 5
SEQ ID NO 7: nucleotide sequence of catalytic domain of GnT-IVb from Homo sapiens
SEQ ID NO 8: amino acid sequence of SEQ ID NO: 7
SEQ ID NO 9: nucleotide sequence of catalytic domain of GnT-Va from Homo sapiens
SEQ ID NO 10: amino acid sequence of SEQ ID NO: 9
SEQ ID NO 11: nucleotide sequence of hybrid GnT-IVa (xylosyltransferase localization signal derived from A. thaliana fused to catalytic domain of GnT-IVa derived from Homo sapiens), codon optimized for Nicotiana benthamiana
SEQ ID NO 12: amino acid sequence of SEQ ID NO: 11
SEQ ID NO 13: nucleotide sequence of hybrid GnT-IVa (fucosyltransferase B localization signal derived from A. thaliana fused to catalytic domain of GnT-IVa derived from Homo sapiens), codon optimized for Nicotiana benthamiana
SEQ ID NO 14: amino acid sequence of SEQ ID NO: 13
SEQ ID NO 15: nucleotide sequence of hybrid GnT-IVb (xylosyltransferase localization signal derived from A. thaliana fused to catalytic domain of GnT-IVb derived from Homo sapiens), codon optimized for Nicotiana benthamiana
SEQ ID NO 16: amino acid sequence of SEQ ID NO: 15
SEQ ID NO 17: nucleotide sequence of hybrid GnT-IVb (fucosyltransferase B localization signal derived from A. thaliana fused to catalytic domain of GnT-IVb derived from Homo sapiens), codon optimized for Nicotiana benthamiana
SEQ ID NO 18: amino acid sequence of SEQ ID NO: 17
SEQ ID NO 19: nucleotide sequence of hybrid GnT-Va (xylosyltransferase localization signal derived from A. thaliana fused to catalytic domain of GnT-Va derived from Homo sapiens), codon optimized for Nicotiana benthamiana
SEQ ID NO 20: amino acid sequence of SEQ ID NO: 19
SEQ ID NO 21: nucleotide sequence of hybrid GnT-Va (fucosyltransferase B localization signal derived from A. thaliana fused to catalytic domain of GnT-Va derived from Homo sapiens), codon optimized for Nicotiana benthamiana
SEQ ID NO 22: amino acid sequence of SEQ ID NO: 21
SEQ ID NO 23: nucleotide sequence of aranesp (human erythropoietin)-codon optimized for Nicotiana benthamiana
SEQ ID NO 24: amino acid sequence of SEQ ID NO: 24
SEQ ID NO 25-48: primer sequences as indicated in the examples

### EXAMPLES

### 1. Expression Constructs for Transient Infiltrations of Chimeric Human GnT-IVa, GnT-IVb and GnT-V in Nicotiana benthamiana

Several hybrid expression constructs were generated based on plant localization signals in combination with a catalytic domain of GnT-IV or GnT-V.

The localization signals (LS) were cloned into the TMV-based 5′ module pICH29590 and the catalytic domains (CD) were cloned into the TMV-based 3′ module cloning vector pICH21595 (Marillonnet et al. (2005) Nature Biotechnology 23, 718-723).
The xylosyltransferase localization signal (XylT LS) was amplified from the full length A. thaliana xylosyltransferase (XylT) gene (clone U13462 obtained from ABRC) using the forward primer: CT XylT FW (5′-caccggtctcaaatg agtaaacggaatccgaag-3′, SEQ ID NO: 25) and reverse primer: CT XylT Rev (5′-caccggtctcatacccgatgagtgaaaaacgaagta-3′), SEQ ID NO: 26). The resulting PCR product of 123 bp, comprising SEQ ID NO: 1, was cloned into pCR2.1-TOPO (Invitrogen) and subsequently digested with the restriction enzyme Bsal and ligated into the Bsal sites of pICH29590 to create pTBN003.
The fucosyltransferase B localization signal (FucTB LS) was amplified from the full length A. thaliana fucosyltransferase B (FucTB) gene (clone U16327 obtained from ABRC) using the forward primer: CT FucTB FW (5′-caccggtctcaaatgggtgttttctcgaatcttc-3′), SEQ ID NO: 27, and reverse primer: CT FucTB Rev (5′-caatggtctcataccgagccgacccagaaacccga-3′), SEQ ID NO: 28. The resulting PCR product of 123 bp, comprising SEQ ID NO: 3, was cloned into pCR-Blunt II-TOPO and subsequently digested with the restriction enzyme Bsal and ligated into the Bsal sites of pICH29590 to generate pTBN004.
The catalytic domain (CD) of GnT-IVa (1527 bp) was amplified from cDNA of human HepG2 cells using the forward primer: CD GnT-IVa FW (5′-caccggtctcaaggtcaaaatgggaaagaaaaactgatt-3′), SEQ ID NO: 29, and reverse primer CD GnT-IVa Rev (5′-caccggtctcaaagctcagttggtggcttttttaatatg-3′), SEQ ID NO: 30. The resulting PCR product (comprising SEQ ID NO: 5) was cloned into pCR-Blunt II-TOPO and subsequently Bsal digested and ligated into the Bsal sites of pICH21595 generating pTBN011.
The CD of GnT-IVb (1548 bp) was amplified from human placental cDNA using the forward primer: CD GnT-IVb FW (5′-caacggtctcaaggtgacgttgtggacgtttaccag-3′), SEQ ID NO: 31, and reverse primer CD GnT-IVb Rev (5′-caccggtctcaaagcttagtcggcctttttcaggaa-3′), SEQ ID NO: 32. The resulting PCR product (comprising SEQ ID NO: 7) was digested with Bsal and ligated into the Bsal sites of pICH21595 generating pTBN010.
The CD of GnT-Va (2109 bp) was amplified from Nicotiana benthamiana codon optimized full length GnT-Va (syn1xVa; see further in example 3 “synthesis of expression constructs for stable transformations of human GnT-IVa, GnT-IVb and GnT-V in Arabidopsis thaliana and Nicotiana benthamiana”) using the forward primer: CD GnT-Va FW (5′-caacggtctcaaggtcctgagtcatcttctatgctc-3′), SEQ ID NO: 33, and reverse primer CD GnT-Va Rev (5′-caacggtctcaaagctcagaggcaatccttacagag-3′), SEQ ID NO: 34. The resulting PCR product (comprising SEQ ID NO: 9) was cloned into pCR4-TOPO and subsequently digested with Bsal and ligated into the Bsal sites of pICH21595, generating pTBN015.

The resulting 3′ and 5′ provectors were subsequently transformed into the Agrobacterium tumefaciens strain GV3101(pMP90) for transient infiltrations in Nicotiana benthamiana.

### 2. Generation of Tri-antennary N-glycans on Endogenous Proteins of WT and XylT/FucT-RNAi N. benthamiana Plants

Nicotiana benthamiana plants with a reduced expression of xylosyltransferase and a reduced expression of fucosyltransferase (further herein designated as XylT/FucT RNAi plants as described in WO2008141806) and also wild type N. benthamiana plants were used to transiently express hybrid GnT-IVa, IVb and Va in combination with XylT or FucTB localization signals (these 6 different hybrid combinations are: xylGnT-IVa, fucGnT-IVa, xylGnT-IVb, fucGnT-IVb, xylGnT-Va and fucGnT-Va). For this, six combinations of LS-CD (Table 1) were used to agro-infiltrate the plants (Marillonnet et al. (2005) Nature Biotechnology 23, 718-723).

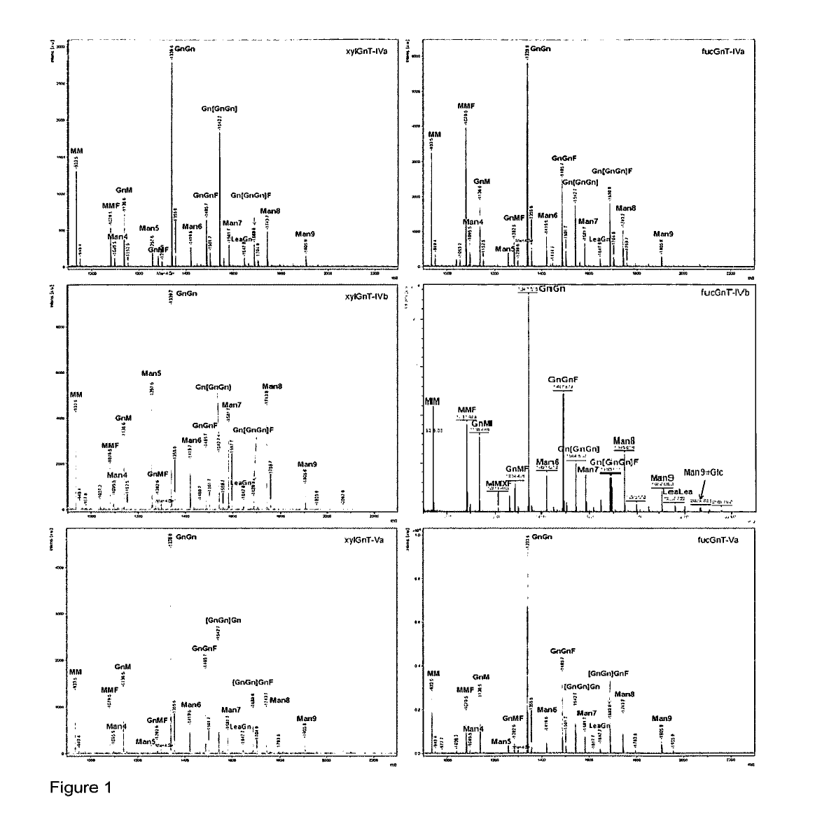

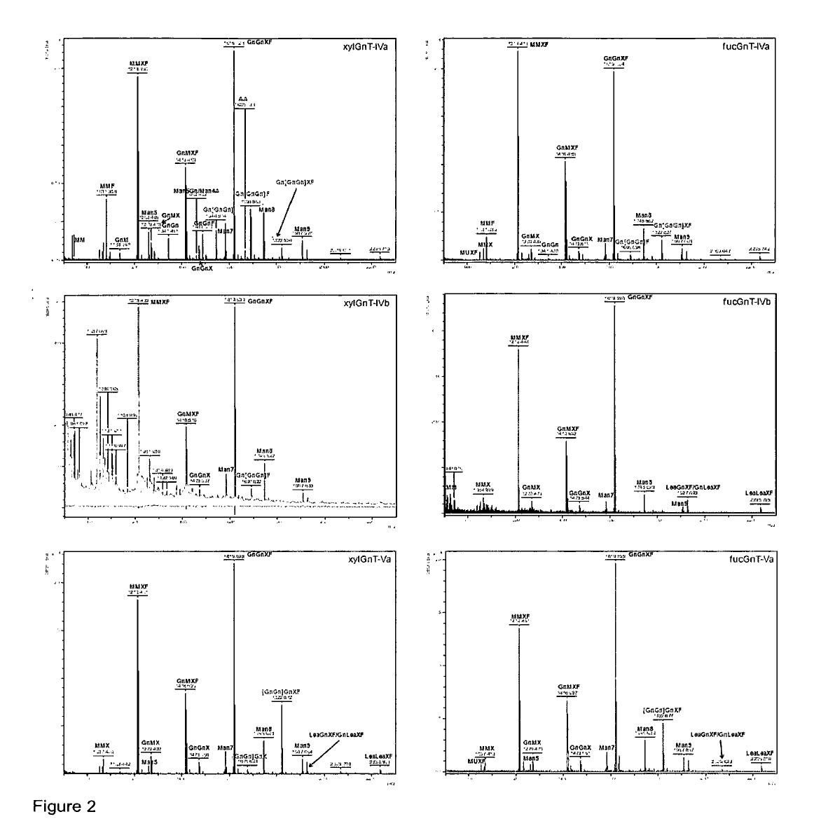

Ten days after infiltration, the transfected leafs were harvested. The endogenous proteins of the infiltrated leafs were analyzed for their N-glycan content using matrix-assisted laser desorption ionization-time of flight mass spectrometry (MALDI-TOF MS) as outlined in Kolarich et al. (2000) Analytical Biochemistry 285, 64-75. Results of this analysis are presented in FIG. 1, FIG. 2 and Table 2.

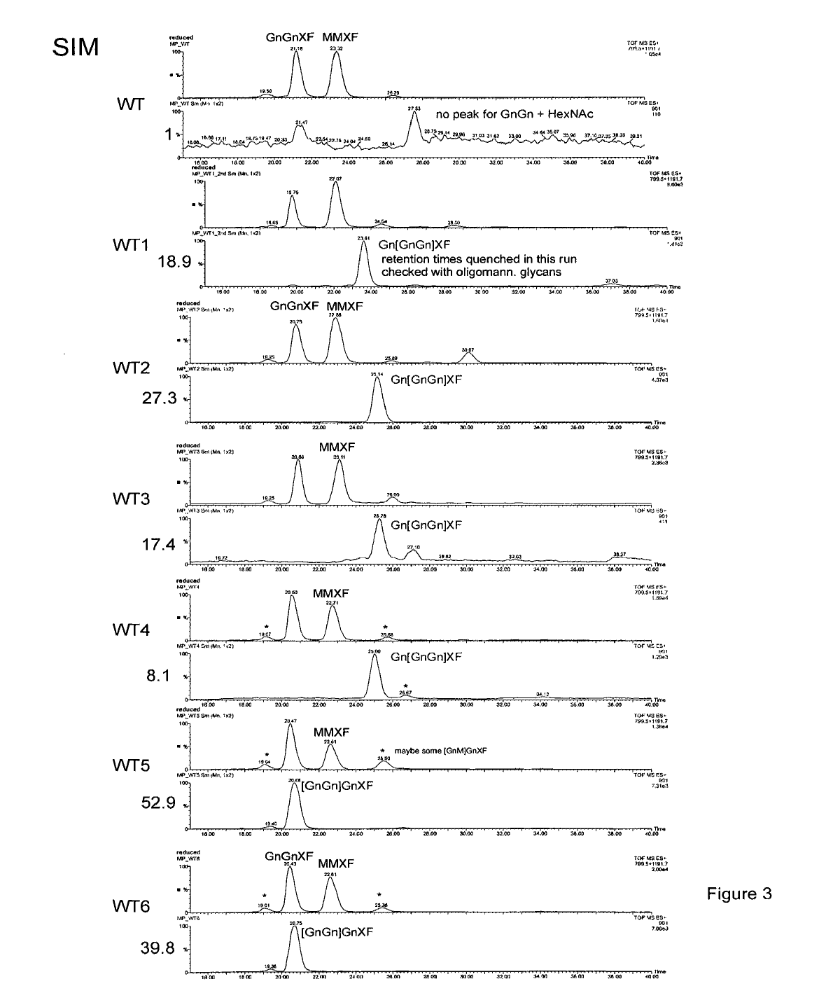

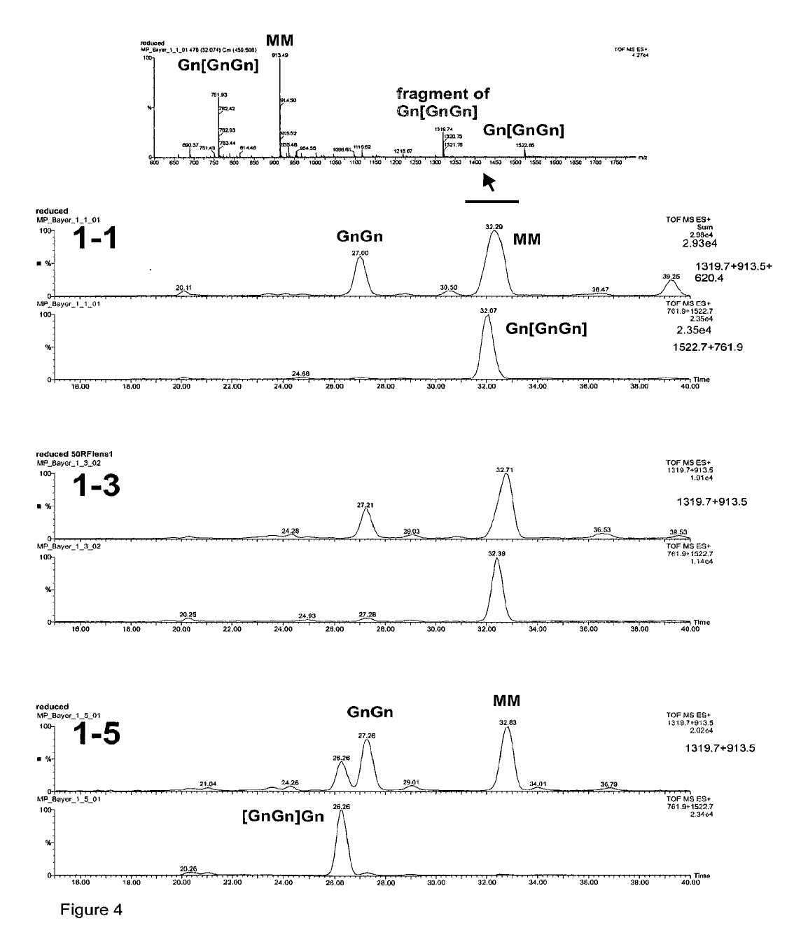

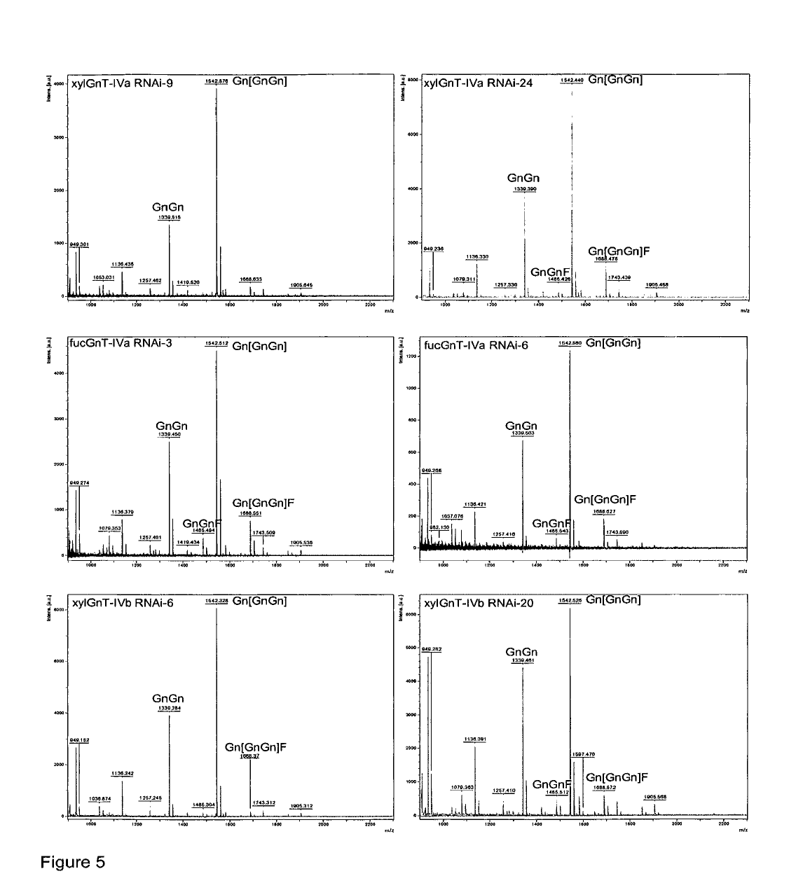

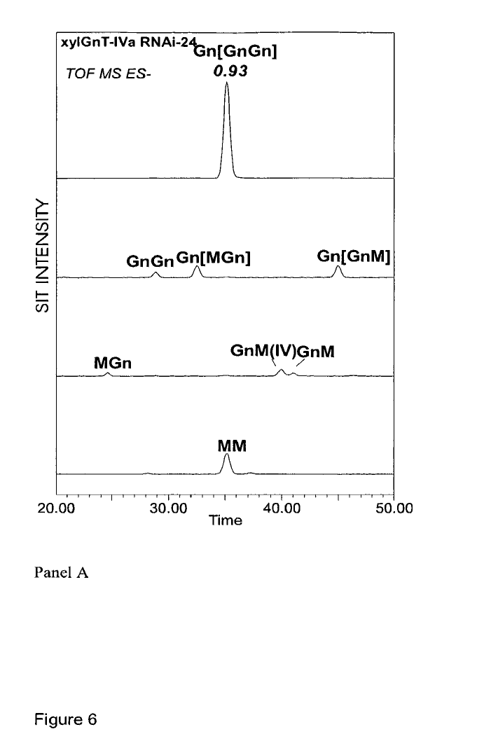

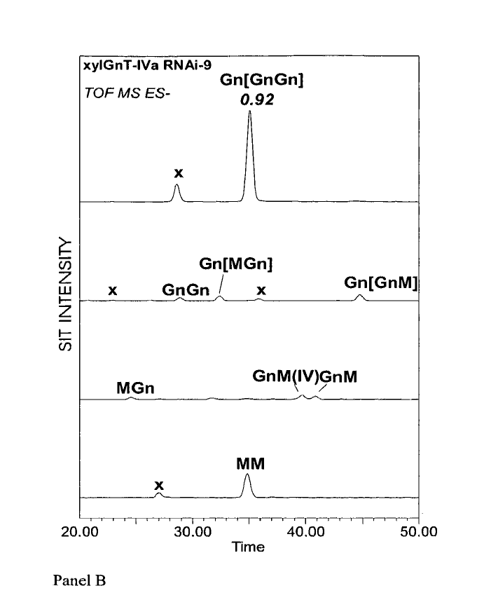

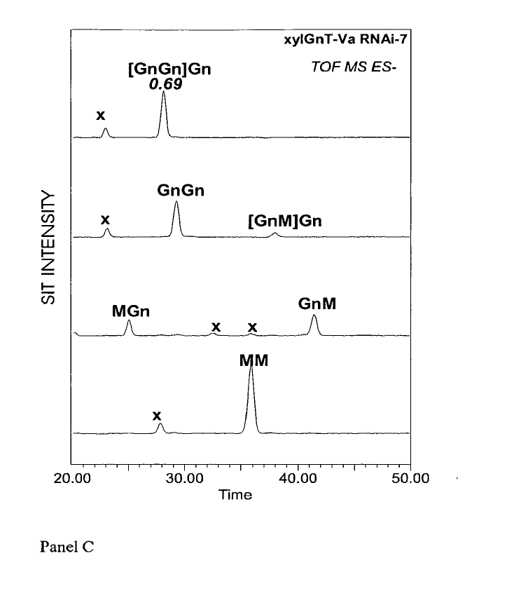

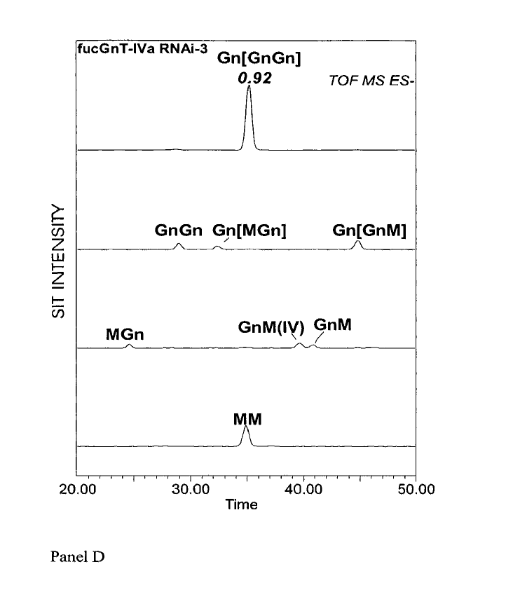

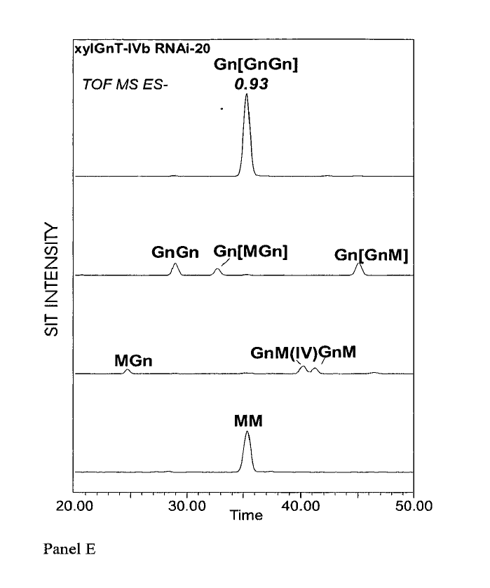

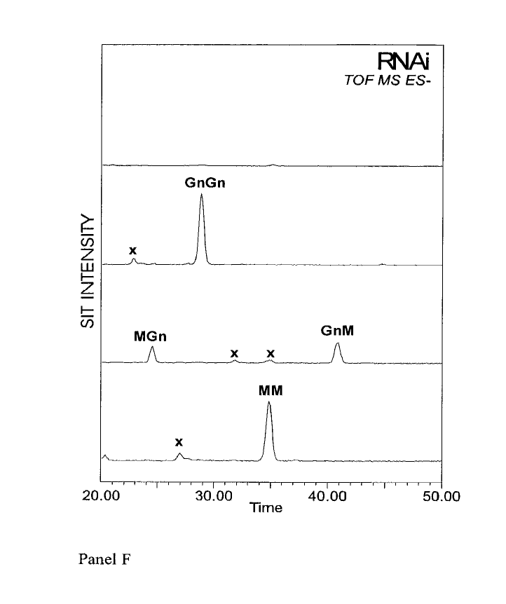

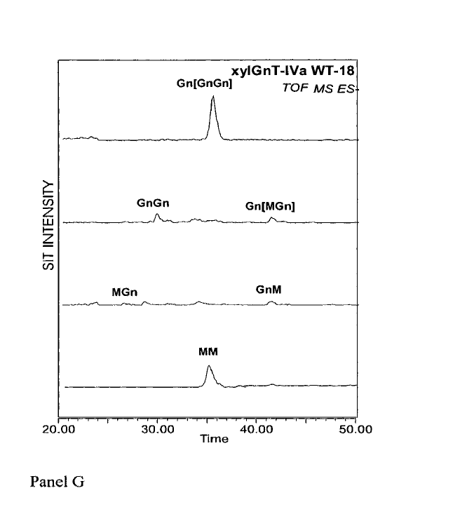

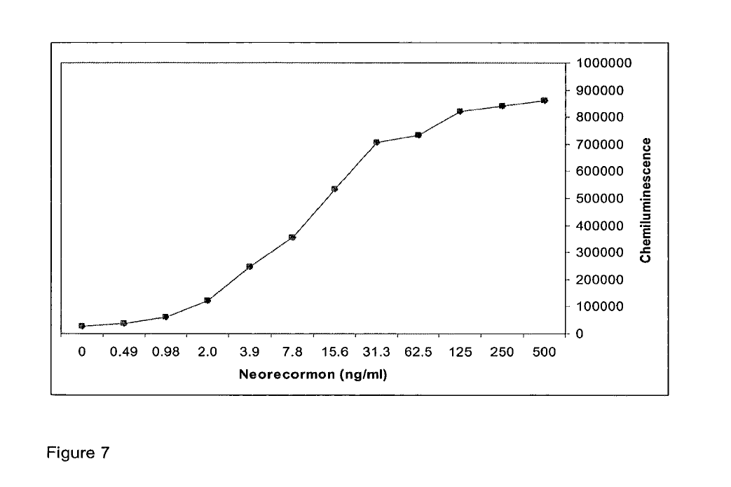

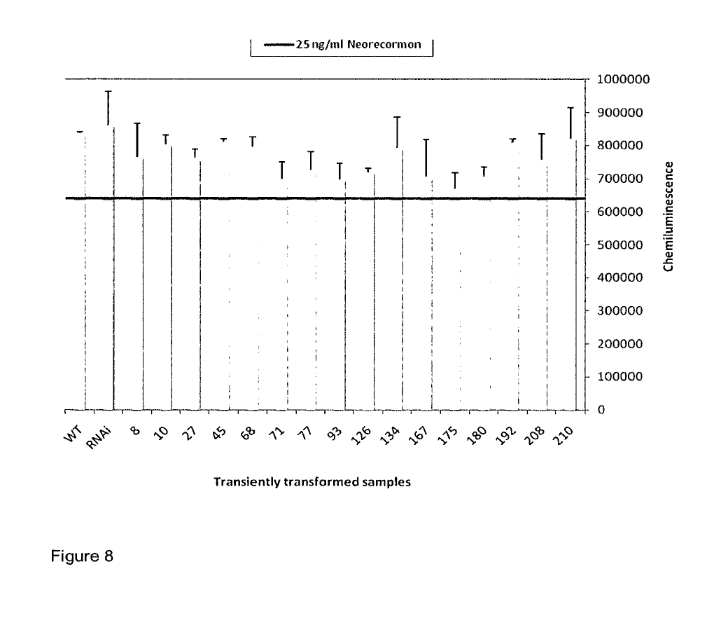

No distinction can be made with MALDI-TOF MS analysis between tri-antennary or bisecting N-glycans in the case of tri-antennary glycans (depicted as GnGnGn-glycans) nor whether bi-antennary glycans (depicted as GnGn-glycans) are GlcNAcβ1-2Manα1-6(GlcNAcβ1-2Manα1-3)Manβ1-4GlcNAcβ1-4GlcNAc-Asn, GlcNAcβ1-6Manα1-6(GlcNAcβ1-2Manα1-3)Manβ1-4GlcNAcβ1-4GlcNAc-Asn or GlcNAcβ1-2Manα1-6(GlcNAcβ1-4Manα1-3)Manβ1-4GlcNAcβ1-4GlcNAc-Asn. To determine the exact composition of the mass peaks representing tri-antennary (GnGnGn-) and bi-antennary (GnGn-glycans) liquid chromatography electrospray ionisation tandem mass spectrometry (LC-ESI MS) was performed according to Stadlmann et al. (2008) Proteomics 8, 2858-2871. Results are shown in FIGS. 3 and 4 and confirm the presence of tri-antennary N-glycans in the infiltrated leaf samples.

In addition, our data clearly show the difference between the linkage of the introduced GlcNAc by the difference in elution time for samples of GnT-IV infiltration in comparison with GnT-V infiltration. Upon GnT-IV expression the tri-antennary glycans (GnGnGn-glycans) were GlcNAcβ1-2Manα1-6(GlcNAcβ1-2(GlcNAc β1-4)Manα1-3)Man β1-4GlcNAcβ1-4GlcNAc-Asn. Upon GnT-V expression the tri-antennary glycans (GnGnGn-glycans) were GlcNAcβ1-6(GlcNAcβ1-2)Manα1-6(GlcNAcβ1-2Manα1-3)Man β1-4GlcNAcβ1-4GlcNAc-Asn.

TABLE 1
Overview of provector combinations to obtain all possible hybrid products
for GnT-IVa, -IVb and Va localization

| | Localization Signal | Catalytic Domain |
|---|---|---|
| Fusion product | 5′ provector | 3′ provector |
| xylGnT-IVa | pTBN003 | pTBN011 |
| fucGnT-IVa | pTBN004 | pTBN011 |
| xylGnT-IVb | pTBN003 | pTBN010 |
| fucGnT-IVb | pTBN004 | pTBN010 |
| xylGnT-Va | pTBN003 | pTBN013 |
| fucGnT-Va | pTBN004 | pTBN013 |

TABLE 2
MALDI-TOF MS analysis of the N-glycans on endogenous proteins of
transfected wild type and XylT/FucT RNAi N. benthamiana plants.
For all combinations localization signal-catalytic domain the relative
abundance of bi-(GnGn) and tri-antennary (GnGnGn) N-glycans are given.

| % | |
|---|---|
| WT | RNAi |

| | GnGn | GnGnGn | GnGn | GnGnGn |
|---|---|---|---|---|
| xylGnT- | 31.4 | 10.6 | 34.2 | 21.3 |
| IVa | | | | |
| fucGnT- | 30.8 | 3.8 | 31.8 | 13.5 |
| IVa | | | | |
| xylGnT- | 35.8 | 2.2 | 27.6 | 6.9 |
| IVb | | | | |
| fucGnT- | 43.6 | 0 | 39.5 | 9 |
| IVb | | | | |
| xylGnT- | 34.9 | 10.9 | 37.8 | 20.4 |
| Va | | | | |
| fucGnT- | 39.3 | 8 | 43.1 | 11.5 |
| Va | | | | |

The obtained data showed that all combinations of localization signal-catalytic domain led to the production of tri-antennary N-glycans in both wild type and RNAi N. benthamiana plants except for fucGnT-IVb in wild type background. When comparing the transfections in WT and RNAi background it is clear that the activity of GnT-IV and GnT-V was more efficient in the RNAi plants. Comparing the different constructs, it is shown that the constructs with the XylT localization signal lead to a higher relative amount of tri-antennary N-glycans as compared to the FucTB signal except for xylGnT-IVb in a XylT/FucT RNAi background. Furthermore, the constructs with the GnT-IVa catalytic domain are slightly more optimal than the ones with the GnT-IVb catalytic domain in terms of producing tri-antennary N-glycans.

The results obtained by these transient infiltrations show that it is possible to introduce tri-antennary N-glycans in N. benthamiana plants, both XylT/FucT RNAi and wild type, by introducing the human genes encoding GnT-IVa, GnT-IVb and GnT-V in combination with the A. thaliana XylT or FucTB localization signal. These data are obtained with the magnICON provector system for testing different hybrid glucosaminyltransferases IV and V.

In a next step we investigated the activity of GnT-IV and -V in stably transformed plants. Therefore, the same combinations as presented in Table 2 are stably expressed into Arabidopsis thaliana WT and XylT/FucTB KO (further referred to as triple KO plants as described in Strasser et al (2004) FEBS Letters 561, 132-136) and also in Nicotiana benthamiana WT and XylT/FucT RNAi plants.

### 3. Synthesis of Expression Constructs for Stable Transformations of Chimeric Human GnT-IVa, GnT-IVb and GnT-V in Arabidopsis thaliana and Nicotiana benthamiana

For stable expression of GnT-IVa, GnT-IVb and GnT-Va in A. thaliana (WT and triple KO) and N. benthamiana (WT and RNAi) plants with localization signals 6 synthetic constructs were made as represented in SEQ ID NO: 11, 13, 15, 17, 19 and 21). All constructs were optimized with the optimal codon use of N. benthamiana (http://www.kazusa.or.jp/codon/cgi-bin/showcodon.cgi?species.4100). The synthetic hybrid fusions were cloned into a plant expression T-DNA vector, containing glyphosate tolerance, under control of a CAMV 35S promoter. SEQ ID NO: 11, 15 and 19 contain the XylT LS fused to the CD of GnT-IVa, -IVb and -Va respectively. These constructs were cloned into the T-DNA vector by XhoI/Mfel digestion and subsequent ligation into the XhoI/EcoRI sites of the T-DNA vector generating pTBN017, pTBN021 and pTBN025 respectively. SEQ ID NO: 13, 17 and 21 contain the FucTB LS fused to a small 5′ part of the GnT-IVa, GnT-IVb and GnT-Va CD. All three are engineered in a way that the XylT LS of pTBN017, pTBN021 and pTBN025 can be exchanged by the FucTB LS of these constructs. SEQ ID NO: 13 and 21 were Mlul/Mfel digested and ligated into the Mlul/EcoRI sites of pTBN017 and pTBN025 generating pTBN019 and pTBN027 respectively while SEQ ID NO: 17 was AvrII/Mfel digested and ligated into the AvrII/EcoRI sites of pTBN021 generating pTBN023. The resulting recombinant vectors are transformed into the Agrobacterium tumefaciens strain C58C1Rif(pGV4000) for stable transformation in Arabidopsis thaliana and into strain C58C1Rif(pGV3000) for stable transformation in Nicotiana benthamiana.

### 4. Generation of Tri-Antennary N-Glycans on Endogenous Proteins of WT and XylT/FucT Knock Out A. thaliana Plants

XylT/FucT knock out (triple KO) and wild type A. thaliana plants were transformed with an aim to obtain stably expressed human GnT-IVa, -IVb and -Va under the A. thaliana XylT and FucTB localization signals (xylGnT-IVa, fucGnT-IVa, xylGnT-IVb, fucGnT-IVb, xylGnT-Va and fucGnT-Va). For this, the above possible hybrid combinations of glucosaminyltransferase IVa, IVb and V (2 different LS in combination with 3 different CD) were used to transform the plants. All plants were transformed via floral dipping (Clough and Bent (1998) The Plant Journal 16, 735-743).

### 5. Generation of Tri-Antennary N-Glycans on Endogenous Proteins of WT and XylT/FucT RNAi N. benthamiana Plants

XylT/FucT RNAi (triple KO) and wild type N. benthamiana plants are used to stably express human GnT-IVa, -IVb and -Va under the A. thaliana XylT and FucTB localization signals (xylGnT-IVa, fucGnT-IVa, xylGnT-IVb, fucGnT-IVb, xylGnT-Va and fucGnT-Va). For this, the above 6 hybrid combinations of glucosaminyltransferase IVa, IVb and V (2 different LS in combination with 3 different CD) were used to transform the plants. All plants were transformed via leaf disk transformation (Regner et al. (1992) Plant Cell Reports 11, 30-33). Glyphosate resistant plants were screened by Real-time PCR to confirm genomic insertion of the hybrid GnT constructs and identify single copy plants. Real-time PCR was performed on genomic DNA with the TaqMan® Universal PCR Master mix (Applied Biosystems, Foster City, Calif.) using the 7500 Fast Real-Time PCR System (Applied Biosystems). In each real-time run a primer set and a probe for the target construct as well as a primer set and a probe for the endogenic control, N. benthamiana XylTg19b gene, were used. In every set of analyzed samples two single copy references and one WT sample were used as control samples. The amplification data were processed with the 7500 Fast System SDS software. The following primer-probe sets were used: target primers and probe directed against the glyphosate resistance gene region (FW epsps: 5′ tcttgctgtggttgccctc 3′ (SEQ ID NO: 35), Rev: 5′ ccaggaagccacgtctctga 3′ (SEQ ID NO: 36), epsps probe: 5′ FAM-ttgccgatggcccgacagc-TAMRA (SEQ ID NO: 37) and endogenic primers and probe (FW XylTg19b: 5′ gcctctctgcccttttggat 3′ (SEQ ID NO: 38), Rev: 5′ aaaggcatttactcgaattacaacaa 3′ (SEQ ID NO: 39) and XylTg19b probe: 5′ VIC-tacgtgtaccatccccagaccccactc-TAMRA (SEQ ID NO: 40). The copy numbers of all samples were calculated by using the 2^{-ΔΔCt} method (Livak et al. 2001). Single copy plants were further analyzed via reverse transcriptase real-time PCR to identify the strongest GnT expressors. Total RNA was isolated from all single copy plants using the RNeasy Plant Mini Kit (Qiagen) and treated with RNase free DNase (Qiagen) to eliminate genomic DNA contamination. The prepared RNA samples (1 μg) were used for the reverse transcriptase reaction using the High-Capacity cDNA Archive Kit (Applied Biosystems). Relative real-time PCR was performed, using the 7500 Fast Real-Time PCR System (Applied Biosystems), on the prepared cDNA with the SYBR Green PCR Master Mix (Applied Biosystems). The N. benthamiana elongation factor 1α (EF1α) gene was used as endogenous control to normalize the amount of cDNA. To process the amplification data, the 7500 Fast System SDS software was used. The expression levels were calculated relative to a WT, not transformed, sample. Following primer combinations were used: endogenic control primers (EF1a FW: 5′ gctgactgtgctgtcctgattatt 3′ (SEQ ID NO: 41), EF1α Rev: 5′ tcacgggtctgtccatcctta 3′ (SEQ ID NO: 42), GnT-IVa target primers (FW: 5′ acaagcctgtgaatgttgagag 3′ (SEQ ID NO: 43), Rev: 5′ cacctggatgttcttgattacc 3′ (SEQ ID NO: 44), GnT-IVb target primers (FW: 5′ ccaacagttttccatcatcttc 3′ (SEQ ID NO: 45) Rev: 5′ actctaacagcaggttgcaatg 3′ (SEQ ID NO: 46) and GnT-Va target primers (FW: 5′ tgcaccacttgaagctattg 3′ (SEQ ID NO: 47) and Rev: 5′ aatcggtgttcttgcttgac 3′ (SEQ ID NO: 48).

Leaves of the best GnT-expressing transformed plants were harvested and the N-glycans of endogenous proteins were subjected to MALDI-TOF-MS to identify and quantify all glycan structures of the stably transformed plants. Results of this analysis are presented in table 3, FIGS. 5 and 6.

TABLE 3
Summarized results of the MALDI-TOF MS N-glycan analysis of stably
transformed wild type and XylT/FucT RNAi N. benthamiana plants
(different transformation events are shown in the Table, column 1).
Column 5 indicates the background: WT (wild type background) or RNAi
(XylT and FucT downregulated). For all samples the relative abundance of
bi-(GnGn), tri-antennary (GnGnGn) and fucosylated N-glycans are given.

| | % | | | |
|---|---|---|---|---|
| Sample | GnGn | % GnGnGn | % fucoslyated | Background |
| WT | 28.98 | 0.00 | 64.70 | WT |
| RNAi | 38.85 | 0.00 | 17.29 | RNAi |
| xylGnT-IVa - 24 | 22.67 | 48.80 | 12.14 | RNAi |
| xylGnT-IVa - 9 | 18.64 | 52.06 | 4.49 | RNAi |
| xylGnT-IVa - 18 | 26.93 | 6.85 | 63.08 | WT |
| xylGnT-IVa - 13 | 36.41 | 4.47 | 71.30 | WT |
| fucGnT-IVa - 8 | 32.69 | 4.28 | 70.41 | WT |
| fucGnT-IVa - 18 | 37.78 | 3.54 | 77.04 | WT |
| fucGnT-IVa - 3 | 23.80 | 40.86 | 13.37 | RNAi |
| fucGnT-IVa - 6 | 23.26 | 45.10 | 11.44 | RNAi |
| xylGnT-IVb - 10 | 23.64 | 0.00 | 45.81 | WT |
| xylGnT-IVb - 19 | 31.13 | 2.93 | 74.51 | WT |
| xylGnT-IVb - 6 | 27.64 | 1.57 | 54.10 | WT |
| xylGnT-IVb - 20 | 23.48 | 30.05 | 7.32 | RNAi |
| xylGnT-IVb - 6 | 23.12 | 45.17 | 3.09 | RNAi |
| fucGnT-IVb - 14 | 27.62 | 0.00 | 58.72 | WT |
| fucGnT-IVb - 6 | 34.64 | 0.00 | 71.05 | WT |
| fucGnT-IVb - 14 | 28.69 | 11.43 | 4.21 | RNAi |
| fucGnT-IVb - 6 | 28.15 | 17.14 | 14.65 | RNAi |
| xylGnT-Va - 6 | 31.27 | 1.87 | 69.67 | WT |
| xylGnT-Va - 8 | 34.13 | 2.00 | 69.41 | WT |
| xylGnT-Va - 1 | 30.53 | 2.24 | 61.98 | WT |
| xylGnT-Va - 17 | 47.47 | 8.17 | 13.77 | RNAi |
| xylGnT-Va - 7 | 34.40 | 19.78 | 14.63 | RNAi |
| fucGnT-Va - 13 | 40.95 | 0.00 | 75.46 | WT |
| fucGnT-Va - 1 | 37.87 | 0.00 | 87.91 | WT |
| fucGnT-Va - 22 | 32.24 | 0.00 | 63.20 | WT |
| fucGnT-Va - 25 | 48.18 | 1.51 | 8.12 | RNAi |
| fucGnT-Va - 13 | 47.78 | 1.31 | 7.07 | RNAi |

When comparing the activity and efficiency of each construct in a WT and RNAi background, it is clear that the RNAi background is more suitable for expression of the human hybrid GnTs since the RNAi background leads to approximately a 10-fold increase of produced tri-antennary N-glycans compared to the WT background. The data also indicate an effect of the localization signal; for all constructs and backgrounds, the hybrid GnTs yield a higher percentage of tri-antennary N-glycans when fused to the XylT LS. On the level of the GnTs itself, the GnT-IVa constructs score best, followed by GnT-IVb and GnT-Va constructs. The most abundant N-glycan structure in samples xylGnT-IVa RNAi-9, xylGnT-IVa RNAi-24, fucGnT-IVa RNAi-3, fucGnT-IVa RNAi-6, xylGnT-IVb RNAi-6 and xylGnT-IVb RNAi-20 is the tri-antennary N-glycan structure (FIG. 5). Furthermore, the glycosylation pattern of xylGnT-IVa RNAi-9 exhibits only two abundant glycan varieties, bi-antennary and tri-antennary N-glycans, and no undesired high-mannose or hybrid N-glycan structures. To confirm the specific activity of the different GnTs, LC-ESI MS analysis was performed and showed the expected linkages of the produced tri-antennary N-glycans of all hybrid GnTs: Gn[GnGn] being glycans with the GlcNAcβ1-2Manα1-6(GlcNAcβ1-2(GlcNAcβ1-4)Manα1-3)Man β1-4GlcNAcβ1-4GlcNAc-Asn conformation) for xylGnT-IVa, fucGnT-IVa, xylGnT-IVb and fucGnT-IVb, and [GnGn]Gn (being glycans with the GlcNAcβ1-6(GlcNAcβ1-2)Manα1-6(GlcNAcβ1-2Manα1-3)Man β1-4GlcNAcβ1-4GlcNAc-Asn conformation) for xylGnT-Va and fucGnT-Va. Results of xylGnT-IVa RNAi-9 and -24, fucGnT-IVa RNAi-3, xylGnT-IVb RNAi-20, xylGnT-Va RNAi-7 and xylGnT-IVa WT-18 are displayed in FIG. 6.

### 6. Generation of Tetra-Antennary N-Glycans on Endogenous Proteins of WT and XylT/FucT RNAi N. benthamiana Plants

To obtain tetra-antennary N-glycans in XylT/FucT RNAi and wild type N. benthamiana plants a combination of two different chimeric genes (one for glucosaminyltransferase IV and the second one for glucosaminyltransferase V) is used (for example XylT LS+GnT-IVa CD and FuCTB LS+GnT-Va CD). For this, the two different GnT coding sequences (GnT-IV and GnT-V) are expressed from “non-competing” viral vectors (as outlined in Giritch et al. (2006) Proc. Natl. Acad. Sc. USA 103, 14701-14706). Therefore the localization signals and catalytic domains are cloned into PVX-based provector magnICON modules. The XylT and FucTB localization signal are cloned into a 5′ PVX-based provector and the GnT-Va into a 3′ PVX-based provector. The double combinations of TMV LS-CD with PVX LS-CD (Table 4) are used to agro-infiltrate the plants.

TABLE 4
Overview of TMV/PVX provector combinations to obtain tetra-antennary
N-glycans

| | Localization | Catalytic | Localization | Catalytic |
|---|---|---|---|---|
| | Signal | Domain | Signal | Domain |
| Fusion | 5′ TMV | 3′ TMV | 5′ PVX | 3′ PVX |
| product | provector | provector | provector | provector |
| TMV xylGnT- | LS XylT | CD GnT-IVa | LS FucTB | CD GnT-Va |
| IVa/ | | | | |
| PVX fucGnT- | | | | |
| Va | | | | |
| TMV fucGnT- | LS FucTB | CD GnT-IVa | LS XylT | CD GnT-Va |
| IVa/PVX | | | | |
| xylGnT-Va | | | | |
| TMV xylGnT- | LS XylT | CD GnT-IVb | LS FucTB | CD GnT-Va |
| IVb/PVX | | | | |
| fucGnT-Va | | | | |
| TMVfucGnT- | LS FucTB | CD GnT-IVb | LS XylT | CD GnT-Va |
| IVb/PVX | | | | |
| xylGnT-Va | | | | |
| TMVxylGnT- | LS XylT | CD GnT-IVa | LS XylT | CD GnT-Va |
| IVa/PVX | | | | |
| xylGnT-Va | | | | |
| TMVxylGnT- | LS XylT | CD GnT-IVb | LS XylT | CD GnT-Va |
| IVb/PVX | | | | |
| xylGnT-Va | | | | |

### 7. Generation of Tetra-Antennary N-Glycans on Endogenous Proteins of WT and XylT/FucT RNAi N. benthamiana and WT and XylT/FucT Knock-Out A. thaliana Plants

To obtain stably expressed tetra-antennary N-glycans in XylT/FucT RNAi and wild type N. benthamiana and WT and XylT/FucT knock-out A. thaliana plants the GnT-IV and GnT-V plants obtained in examples 4 and 5 are crossed.

### 8. Synthesis of Expression Constructs for Transient and Stable Expression of the Therapeutic relevant Protein Aranesp into Plants Comprising GnT-IV or GnT-V and into Plants Comprising GnT-IV and GnT-V

In order to demonstrate that tri-antennary and tetra-antennary N-glycans can be produced on recombinant glycoproteins, the human darbepoetin alfa (also designated as Aranesp, which is a synthetic form of human erythropoietin containing two extra N-linked glycosylation acceptor sites as compared to human erythropoietin) is expressed in the plants comprising GnT-IV and in plants comprising both GnT-IV and GnT-V. The coding sequence of Aranesp is synthetically made and codon optimized for expression in N. benthamiana. In addition, the coding sequence comprises an amino-terminal secretion signal peptide and a carboxy-terminal histidine tag for purification of the protein. SEQ ID NO: 23 depicts the Aranesp coding sequence fused to the secretion signal peptide and the his-tag (the first 24 amino acids of SEQ ID NO: 24 correspond with the secretion signal peptide and the last 12 amino acids of SEQ ID NO: 24 correspond with the histidine tag). The resulting construct is cloned into a plant expression vector under control of the Rubisco small subunit promoter.

### 9. Transient Expression of Aranesp into Plants Comprising GnT-IV or GnT-V and into Plants Comprising GnT-IV and GnT-V

GnT-IV and/or GnT-V comprising XylT/FucT RNAi and WT N. benthamiana plants were used to transiently express hybrid Aranesp, via agro-infiltration (Marillonnet et al. (2005) Nature Biotechnology 23, 718-723). Ten days after infiltration the transfected leafs were harvested. The endogenous proteins were extracted and analyzed for presence and quantification of erythropoietin by a commercial available homogeneous immunoassay (Van Maerken et al. (2010) Journal of Applied Physiology doi:10.1152/japplphysio1.01102.2009). Results of these analysis are presented in Table 5 and show that all plants efficiently express hybrid Aranesp.

TABLE 5
Overview of EPO amounts in GnT-IV and GnT-V transgenic
N. benthamiana plants

| | | | | Total | EPO/ |
|---|---|---|---|---|---|
| Sam- | EPO | Epo- | Epo- | protein | total |
| ple | expressing | concentration | concentration | conc | protein |
| nr | samples | mIU/ml | mg/l | (mg/l) | (%) |
| | WT | 70.45 | 0.27 | 686.8 | 0.04 |
| | RNAi | 186.32 | 0.72 | 958.1 | 0.07 |
| 8 | xylGnT-Va | 223.6 | 0.86 | 803.4 | 0.11 |
| | RNAi | | | | |
| 10 | xylGnT-Va | 118.97 | 0.46 | 715.2 | 0.06 |
| | RNAi | | | | |
| 27 | fucGnT-IVa | 85.29 | 0.33 | 756.6 | 0.04 |
| | RNAi | | | | |
| 45 | fucGnT-IVa | 478.93 | 1.84 | 979.8 | 0.19 |
| | RNAi | | | | |
| 68 | fucGnT-IVa | 133.39 | 0.51 | 435.7 | 0.12 |
| | RNAi | | | | |
| 71 | fucGnT-IVa | 98.51 | 0.38 | 564.3 | 0.07 |
| | RNAi | | | | |
| 77 | xylGnT-IVb | 195.29 | 0.75 | 698.8 | 0.11 |
| | RNAi | | | | |
| 93 | xylGnT-IVb | 137.75 | 0.53 | 736.9 | 0.07 |
| | RNAi | | | | |
| 126 | xylGnT-IVa | 272.09 | 1.05 | 1212.5 | 0.09 |
| | RNAi | | | | |
| 134 | xylGnT-IVa | 132.4 | 0.51 | 381.2 | 0.13 |
| | RNAi | | | | |
| 167 | xylGnT-IVa | 464.46 | 1.79 | 727.2 | 0.25 |
| | WT | | | | |
| 175 | xylGnT-IVa | 455.71 | 1.75 | 842.9 | 0.21 |
| | WT | | | | |
| 180 | fucGnT-IVa | 129.25 | 0.5 | 648.7 | 0.08 |
| | WT | | | | |
| 192 | fucGnT-IVa | 300.79 | 1.16 | 957.6 | 0.12 |
| | WT | | | | |
| 208 | xylGnT-Va | 73.15 | 0.28 | 293.5 | 0.1 |
| | WT | | | | |
| 210 | xylGnT-Va | 217.39 | 0.84 | 871.8 | 0.1 |
| | WT | | | | |
| | non EPO | 0 | 0 | 3763 | 0.00 |
| | expressing | | | | |
| | WT | | | | |
| | samples | | | | |

The activity of the introduced Aranesp protein was tested using an in vitro assay. For this assay HEK293T cells were transfected with a chimeric EpoR-mLR-FFY receptor (Zabeau et al. (2004) Molecular Endocrinology 18 (1) 150-161) and the STAT3 responsive rPAP1-luciferase reporter. Overnight incubation of the cells with a defined amount of plant produced Aranesp or commercially available Neorecormon stimulates the chimeric EpoR-mLR-FFY receptor, generating a STAT3 signal. The STAT3 responsive rPAP1-luciferase reporter makes it possible to quantify the Aranesp activity by measuring the chemoluminescence. FIGS. 7 and 8 show the results for a serial dilution of the commercially available Neorecormon and for a 25 ng/ml dilution of the plant produced hybrid Aranesp samples respectively after overnight stimulation of the cells.

The in vitro activity test in which the efficiency of Aranesp binding to the EPO receptor is tested, shows that all transiently transformed N. benthamiana plants produce active Aranesp. Moreover, the activity of the plant produced Aranesp is even higher than the activity which is observed with the same amount of Neorecormon.

1. A method to produce multi-antennary glycoproteins in plant cells comprising the steps of:
a. providing a plant cell with a chimeric gene comprising the following operably linked nucleic acid molecules:
i) a plant-expressible promoter,
ii) a DNA region encoding a functional N-acetylglucosaminyltransferase IV,
iii) a DNA region involved in transcription termination and polyadenylation, and
b. cultivating said plant cell and isolating multi-antennary glycoproteins from said plant cell.

2. The method according to claim 1 further comprising the step of providing the plant cell with a second chimeric gene comprising the following operably linked nucleic acid molecules:
i) a plant expressible promoter,
ii) a DNA region encoding a functional N-acetylglucosaminyltransferase V,
iii) a DNA region involved in transcription termination and polyadenylation.

3. The method according to claim 1 wherein the plant cells have no detectable beta-(1,2)xylosyltransferase and no detectable alfa-(1,3)fucosyltransferase activity.

4. The method according to claim 1 wherein said N-acetylglucosaminyltransferase IV and/or said N-acetylglucosaminyltransferase V are of the mammalian type.

5. The method according to claim 1 wherein said N-acetylglucosaminyltransferase IV and/or said N-acetylglucosaminyltransferase V are of the hybrid type.

6. The method according to claim 1 further introducing a third chimeric gene comprising a plant expressible promoter and a DNA region encoding a beta(1,4)-galactosyltransferase.

7. The method according to claim 1 wherein a heterologous glycoprotein is additionally expressed in said plant cells from a chimeric gene comprising a plant expressible promoter and a DNA region encoding said heterologous glycoprotein.

8. A multi-antennary glycoprotein obtained by the method of claim 1.

9. A plant cell comprising a chimeric gene comprising the following operably linked nucleic acid molecules:
i) a plant-expressible promoter,
ii) a DNA region encoding a functional N-acetylglucosaminyltransferase IV,
iii) a DNA region involved in transcription termination and polyadenylation.

10. The plant cell according to claim 9 further comprising a second chimeric gene comprising the following operably linked nucleic acid molecules:
i) a plant expressible promoter,
ii) a DNA region encoding a functional N-acetylglucosaminyltransferase V,
iii) a DNA region involved in transcription termination and polyadenylation.

11. The plant cell according to claim 9 wherein said plant cell has no detectable beta-(1,2)xylosyltransferase activity and no detectable alfa-(1,3)fucosyltransferase activity.

12. The plant cell according to claim 9 wherein said N-acetylglucosaminyltransferase IV and/or said N-acetylglucosaminyltransferase V are of the mammalian type.

13. The plant cell according to claim 9 wherein said N-acetylglucosaminyltransferase IV and/or said N-acetylglucosaminyltransferase V are of the hybrid type.

14. The plant cell according to claim 9 comprising a heterologous glycoprotein which is expressed in said plant cell from a chimeric gene comprising a plant expressible promoter and a DNA region encoding said heterologous glycoprotein.

15. A plant consisting essentially of the plant cell according to claim 9.
